(19) United States
(12) Patent Application Publication
(10) Pub. No.: US 2025/0136662 A1
(43) Pub. Date: May 1, 2025

(54) CD95 POLYPEPTIDES

(71) Applicants: Deutsches Krebsforschungszentrum Stiftung des öffentlichen Rechts, Heidelberg (DE); Universität Heidelberg, Heidelberg (DE)

(72) Inventors: Ana MARTIN-VILLALBA, Heidelberg (DE); Britta BRÜGGER, Schriesheim (DE); Ilpo VATTULAINEN, Espoo (FI); Fabio LOLICATO, Heidelberg (DE); Gülce Sila GÜLCÜLER, Heidelberg (DE); Susanne KLEBER, Heidelberg (DE)

(73) Assignees: Deutsches Krebsforschungszentrum Stiftung des öffentlichen Rechts, Heidelberg (DE); Universität Heidelberg, Heidelberg (DE)

(21) Appl. No.: 18/713,473

(22) PCT Filed: Nov. 24, 2022
(86) PCT No.: PCT/EP2022/083143

§ 371 Date: May 24, 2024

(30) Foreign Application Priority Data
Nov. 25, 2021 (EP) 21210475.6

(51) Int. Cl. C07K 14/705; A61K 38/00; A61K 45/06

(52) U.S. Cl. CPC C07K 14/70578; A61K 45/06; A61K 38/00

(57) ABSTRACT

The present invention relates to an effector polypeptide comprising (i) an amino acid sequence at least 70% identical to the amino acid sequence RSNLGWLCLLLLPIPLIVWVKRK (SEQ ID NO: 1); and (ii) an exchange of an amino acid to a non-identical amino acid at least one position selected from the list consisting of positions 1, 2, 3, 19, 22, and 23 of the amino acid sequence of (i). The present invention also relates to a polynucleotide comprising a nucleic acid sequence encoding the aforesaid effector polypeptide. and to related host cells. pharmaceutical compositions. and uses. and to related uses in medicine, in particular in treating and/or preventing cancer, inflammatory disease, or acute or chronic neurodegenerative disease.

The present invention relates to an effector polypeptide comprising (i) an amino acid sequence at least 70% identical to the amino acid sequence RSNLGWLCLLLLPIPLIVWVKRK (SEQ ID NO: 1); and (ii) an exchange of an amino acid to a non-identical amino acid at at least one position selected from the list consisting of positions 1, 2, 3, 19, 22, and 23 of the amino acid sequence of (i). The present invention also relates to a polynucleotide comprising a nucleic acid sequence encoding the aforesaid effector polypeptide, and to related host cells, pharmaceutical compositions, and uses, and to related uses in medicine, in particular in treating and/or preventing cancer, inflammatory disease, or acute or chronic neurodegenerative disease.

CD95, also known as Fas Receptor or APO-1, is a transmembrane receptor involved in cell clearing in development and degenerative diseases and CD95-mediated kinase activity is involved in inflammation and cancer progression. CD95 on cancer cells activates migration, invasion, proliferation and cancer metastasis. CD95 on immune cells modulates the immune response, e.g. to cancer cells. In accordance, Asunercept, a soluble CD95-Fc fusion protein which binds to CD95L and thereby prevents binding of CD95L to endogenous CD95, was developed to prevent CD95-mediated signaling induction in activated T-cells and cancer cells e.g. in glioblastoma multiforme or pancreatic ductal adenocarcinoma.

On a cellular level, engagement of CD95 by its ligand (CD95L) can induce either apoptosis or cell survival (Martin-Villalba et al. (2013), Trends Mol Med 19 (6): 329). Apoptosis is initiated by activation of caspases in a death-inducing signaling complex through the interactions at the intracellular death domain of CD95. Phosphorylation of a tyrosine within this domain recruits another complex with kinase activity, which outcompetes the death-inducing complex (Sancho-Martinez & Martin-Villalba (2009); Cell Cycle. 8:838). SH2-domain containing proteins bind to the phosphorylated tyrosine, ultimately leading to increased PI3K or ERK activity, depending on the cell type (Martin-Villalba et al. (2013), loc. cit.). Thus, the cell's fate is governed by a molecular switch controlling the recruitment of either a death-or a kinase-inducing complex to CD95. Notably, in cancer cells, CD95 has no loss-of-function mutations, and efforts to delete this gene in primary cells leads to so-called death by neglect. This feature suggest a CD95L-independent function of CD95 in cell survival, which would evade Asunercept targeting.

CD95's cognate ligand, CD95L, is a Type 2 transmembrane protein that can either be presented on neighboring cells or cleaved and presented as soluble ligand. Signaling is mediated via the trimeric form of CD95L; artificial mimics of trimeric CD95L are known in the art (cf. e.g. Kleber et al. (2008), Cancer Cell 13:235-248). The source of CD95L in cancer can be the cancer cell itself, but also the endothelial and immune cells represent a very important source (cf. e.g. (Martin-Villalba et al. (2013), loc. cit.).

At present, molecular mechanisms governing the decision on apoptosis or cell survival are largely unknown; recently, however, it was found that cell-cell contacts have an impact on CD95's activation mode (Gülcüler Balta (2019), Ph.D. thesis submitted to the University Heidelberg). A general feature of cell-cell contact is the selective compartmentalization of membrane domains favoring a distinct cooperativity of signaling modules (Bethani et al. (2010), EMBO J. 29:2677).

In view of the above, there is still a need in the art for improved means and methods for treatment of diseases in which CD95 signaling is relevant.

This problem is addressed by the effector polypeptides, polynucleotides, host cells, compositions, kits, and uses with the features of the independent claims. Advantageous embodiments which might be realized in an isolated fashion or in any arbitrary combinations are listed in the dependent claims.

In accordance, the present invention relates to an effector polypeptide comprising
(i) an amino acid sequence at least 70% identical to the amino acid sequence RSNLGWLCLLLLPIPLIVWVKRK (SEQ ID NO:1); and
(ii) an exchange of an amino acid to a non-identical amino acid in at least one position selected from the list consisting of positions 1, 2, 3, 19, and 22 of the amino acid sequence of (i).

Also, the present invention relates to an effector polypeptide comprising
(i) an amino acid sequence at least 70% identical to the amino acid sequence RSNLGWLCLLLLPIPLIVWVKRK (SEQ ID NO:1); and
(ii) an exchange of an amino acid to a non-identical amino acid in at least one position selected from the list consisting of positions 1, 2, 3, 19, and 22 of the amino acid sequence of (i).

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof, thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified, in particular the polynucleotides and polypeptides, may be comprised in larger structures, e.g. may be covalently or non-covalently linked to further sequences, carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C-or N-terminally to the fusion polypeptide. Said stretch of amino acids preferably allows for detection of the polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences, e.g. an IL-2 signal sequence, and linker sequences.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually referred to as “peptides”. Preferably, the polypeptide comprises of from 20 to 1000, more preferably of from 21 to 500, still more preferably of from 22 to 400, most preferably of from 23 to 350 amino acids. Preferably, the polypeptide is comprised in a fusion polypeptide and/or a polypeptide complex.

The term “effector polypeptide”, as used herein, relates to a polypeptide comprising the amino acid sequence as specified; thus, the effector polypeptide comprises (i) an amino acid sequence at least 70% identical to the amino acid sequence RSNLGWLCLLLLPIPLIVWVKRK (SEQ ID NO: 1); and (ii) an exchange of an amino acid to a non-identical amino acid in at least one position selected from the list consisting of positions 1, 2, 3, 19, 22, and 23 of the amino acid sequence of (i). Thus, the effector polypeptide is a mutein of a polypeptide comprising the amino acid sequence of SEQ ID NO:1.

Preferably, the effector polypeptide is a non-naturally occurring polypeptide, in particular is an artificial polypeptide; thus, preferably, the effector polypeptide comprises an amino acid sequence not occurring in nature. Thus, the effector polypeptide may comprise one or more artificial amino acids. Preferably, the effector polypeptide is synthesizable by protein biosynthesis; thus, the effector polypeptide comprises, preferably consists of, L-amino acids, preferably L-alpha amino acids, more preferably proteinogenic amino acids.

The sequence at least 70% identical to SEQ ID NO:1 preferably is at least 75% identical to SEQ ID NO: 1, more preferably at least 80%, still more preferably at least 85%, even more preferably at least 90%, most preferably at least 95% identical to SEQ ID NO:1. Preferably, the sequence at least 70% identical to SEQ ID NO:1 corresponds to SEQ ID NO:1 with at least one, preferably at least two, more preferably two, amino acid exchanges, deletions, and/or insertions, preferably amino acid exchanges. As the skilled person understands, an “exchange” of an amino acid in an amino acid sequence relates to a replacement of an amino acid by a non-identical amino acid. Preferably, an exchange of an amino acid is a conservative exchange, i.e. an exchange of an amino acid for a non-identical amino acid of the same functional group of amino acids. Thus, preferably, an amino acid with a hydrophobic side chain (“hydrophobic amino acid”, A, V, L, I, M, F, Y, or W) is exchanged for a non-identical hydrophobic amino acid, also preferably, an amino acid with a negatively charged side chain (D or E), is exchanged for a non-identical amino acid with a negatively charged side chain; also preferably, an amino acid with an uncharged polar side chain (S, T, N, or Q) is exchanged for an amino acid with an uncharged polar side chain; also preferably, an amino acid with a positively charged side chain (R, H, or

K) is exchanged for a non-identical amino acid with a positively charged side chain. Also preferably, in a conservative exchange an amino acid is exchanged for a non-identical amino acid having the same functional group or structurally similar group in the side chain, e.g. —OH or —SH (S, T, C), —COOH (D, E), helix breaking properties (G, P) and the like. Preferably, the amino acid exchange(s) is/are selected from the list consisting of R1Q, R1E, R1A, S2A, N3A, R1E/N3A, R1A/N3A, W19A, R22A, R22E, K23A, K23E, R22A/K23A, R22E/K23E, W19A/R22A/K23A, W19A/R22E/K23E, and any combination thereof; more preferably, the amino acid exchange is R1Q, R1E, or R1A, still more preferably is R1E or K23E.

Preferably, the effector polypeptide comprises the amino acid sequence X¹X²X³LGWLCLLLLPIPLIVX⁴VKX⁵X⁶ (SEQ ID NO:2), wherein X¹ is selected from E, Q, A, N, G, V, L, I, S, T, D, and M, preferably is E or Q; X² is selected from A, G, V, L, and I, X³ is selected from A, G, V, L, I, Q, N, D, E, S, T, M, H, K, and R; and/or X⁴ to X⁶ are independently selected from A, G, V, L, I, Q, N, D, E, S, T, and M. More preferably, the effector polypeptide comprises the amino acid sequence ESNLGWLCLLLLPIPLIVWVKRK (SEQ ID NO: 3), the amino acid sequence QSNLGWLCLLLLPIPLIVWVKRK (SEQ ID NO:4), or the amino acid sequence ASNLGWLCLLLLPIPLIVWVKRK (SEQ ID NO:5).

In a preferred embodiment, the effector polypeptide of any one of claims 1 to 3 comprising at least two, in an embodiment at least three amino acid exchanges. More preferably, said amino acid exchanges are exchanges for acidic amino acids, preferably to glutamic acid (E). In a further preferred embodiment, the amino acid exchange(s) is/are selected from the list consisting R1E, R22E, and K23E, more preferably the effector polypeptide comprises the amino acid exchanges comprise R1E, R22E, and K23E. In a further preferred embodiment, the effector polypeptide comprises the amino acid sequence RSNLGWLCLLLLPIPLIVWVKEE (SEQ ID NO:33) or comprises the amino acid sequence ESNLGWLCLLLLPIPLIVWVKEE (SEQ ID NO:34).

The amino acid sequence of SEQ ID NO:1 corresponds to the transmembrane domain, i.e. amino acids 171 to 193 of human CD95 (Genbank Acc. No: NP_000034.1). Thus, the effector polypeptide may be a CD95 polypeptide or a truncated version thereof having the properties as specified herein, i.e. the structural properties and, preferably at least one activity, as specified herein. Thus, the effector polypeptide may in particular include variants of human CD95 (CD95 variants) having the aforesaid properties. Thus, preferably, the term variant of human CD95, as used herein, relates to a variant comprising an amino acid sequence characterized in that the sequence can be derived from the aforementioned specific amino acid sequence by at least one amino acid substitution, addition and/or deletion, wherein the CD95 variant shall preferably have the activity as specified elsewhere herein. Preferably, CD95 variant is an ortholog, a paralog or another homolog of human CD95; the CD95 variant may, however, also be a chimeric CD95, preferably comprising domains from at least two non-identical species. Further, CD95 variants include variants comprising amino acid sequences which are at least 70%, at least 75%, at least 80%, at least 85%, at least 90%, at least 95%, at least 98%, or at least 99% identical to the amino acid sequence of human CD95. Thus, the effector polypeptide preferably further comprises an amino acid sequence essentially identical, preferably identical, to at least one of the amino acid sequences of SEQ ID NOs: 6 to 10; and/or an amino acid sequence essentially identical, preferably identical, to at least one of the amino acid sequences of SEQ ID NOs: 11 to 13; more preferably, the effector polypeptide further comprises an amino acid sequence essentially identical, preferably identical, to at least one of the amino acid sequences of SEQ ID NOs: 6 to 10 as N-terminal sequence(s); and/or an amino acid sequence essentially identical, preferably identical, to at least one of the amino acid sequences of SEQ ID NOs: 11 to 13 as C-terminal sequence(s), wherein SEQ ID NO:6 has the amino acid sequence KCKEEGS; SEQ ID NO:7 has the amino acid sequence CRCKPNFFCNSTVCEHCDPCTKCEHGIIKECTLTSNT; SEQ ID NO:8 has the amino acid sequence VPCQEGKEYTDKAHFSSKCRRCRLCDEGHGLEVEINCTRTQNT; SEQ ID NO: 9 has the amino acid sequence QNLEGLHHDGQFCHKPCPPGERKARDCTVNGDEPDC; SEQ ID NO:10 has the amino acid sequence MLGIWTLLPLVLTSVARLSSKSVNAQVTDINSKGLELRKTVTTVET; SEQ ID NO:11 has the amino acid sequence EVQKTCRKHRKENQGSHESPTLNPETVAINLSDVDL; SEQ ID NO: 12 has the amino acid sequence SKYITTIAGVMTLSQVKGFVRKNGVNEAKIDEIKNDNVQDTAEQV QLLRNWHQLHGKKEAYDTLIKDLKKANLCTLAEKIQTIILKDI; and SEQ ID NO:13 has the amino acid sequence TSDSENSNFRNEIQSLV. The aforesaid sequences of SEQ ID NOS: 6 to 13 are derived from human CD95, with SEQ ID NO:6 being the internal linker sequence, SEQ ID NO:7 corresponding to the cysteine-rich domain 3 (CRD3) domain, SEQ ID NO:8 corresponding to the CRD2 domain, SEQ ID NO:9 corresponding to the CRD1 domain, SEQ ID NO: 10 corresponding to the signal peptide (SP) and linker regions, SEQ ID NO:11corresponding to the cytosolic domain, SEQ ID NO:12 corresponding to the death domain, SEQ ID NO: 13 corresponding to the C-terminal intracellular domain (CID). More preferably, the effector polypeptide comprises, preferably consists of, in the order of N-terminus to C-terminus, the amino acid sequences of (I) SEQ ID NO:1-SEQ ID NO:11; (II) SEQ ID NO:1-SEQ ID NO: 11-SEQ ID NO:12; (III) SEQ ID NO:1-SEQ ID NO:11-SEQ ID NO:12-SEQ ID NO: 13; (IV) SEQ ID NO:6-SEQ ID NO:1-SEQ ID NO:11-SEQ ID NO:12; (V) SEQ ID NO: 7-SEQ ID NO:6-SEQ ID NO:1-SEQ ID NO:11-SEQ ID NO:12; (VI) SEQ ID NO:8-SEQ ID NO:7-SEQ ID NO:6-SEQ ID NO:1-SEQ ID NO:11-SEQ ID NO:12; (VII) SEQ ID NO: 9-SEQ ID NO:8-SEQ ID NO:7-SEQ ID NO:6-SEQ ID NO:1-SEQ ID NO:11-SEQ ID NO:12; or (VIII) SEQ ID NO:10-SEQ ID NO:9-SEQ ID NO:8-SEQ ID NO:7-SEQ ID NO:6-SEQ ID NO:1-SEQ ID NO:11-SEQ ID NO:12, or a sequence essentially identical to one of the aforesaid sequences. Thus, the effector polypeptide may in particular be a CD95 polypeptide comprising the amino acid exchange(s) as specified herein and lacking the N-terminal extracellular domains, or may be a CD95 polypeptide comprising the amino acid exchange(s) as specified herein. Thus, preferably, the effector polypeptide comprises, more preferably consists of, the amino acid sequence of SEQ ID NO:14 or SEQ ID NO:15. Also preferably, the effector polypeptide comprises, more preferably consists of, the amino acid sequence encoded by a nucleic acid sequence selected from the group consisting of SEQ ID NOs: 16, 18, 21, 24, and 27 according to the standard genetic code.

In a preferred embodiment, the effector polypeptide comprises, more preferably consists of, the amino acid sequence of SEQ ID NO:30, i.e. preferably comprises an R171E exchange. In a further preferred embodiment, the effector polypeptide comprises amino acid exchanges at least in positions 192 and 193 of the CD95 polypeptide; in particular, the effector polypeptide may comprise, more preferably consist of, the amino acid sequence of SEQ ID NO:31, i.e. preferably comprises an R192E and a K193E exchange. In an even more preferred embodiment, the effector polypeptide comprises amino acid exchanges at least in positions 171, 192 and 193 of the CD95 polypeptide; in particular, the effector polypeptide may comprise, more preferably consist of, the amino acid sequence of SEQ ID NO:32, i.e. preferably comprises an R171E, an R192E, and a K193E exchange.

Preferably, the effector polypeptide has the activity of integrating into biological membranes, preferably into biological membranes of mammalian cells, more preferably of host cells as specified elsewhere herein. Preferably, said membrane integration is spontaneous, i.e. if contacted with biological membranes, the effector polypeptide integrates into said biological membranes without requirement for one or more further factor(s). Also preferably, the effector polypeptide integrates into biological membranes by means of biological intracellular trafficking pathways; as will be appreciated, in such case the effector polypeptide preferably comprises a signal peptide. Appropriate signal peptides are known in the art; preferably, said signal peptide comprises the amino acid sequence of SEQ ID NO:10. Also preferably, the effector polypeptide comprises a membrane insertion peptide; corresponding peptides are known in the art, e.g. from Svoronos et al. (2020), Mol Pharmaceutics 17:461. Preferably, at least 10%, more preferably at least 20%, still more preferably at least 50%, of a given amount of the effector polypeptide integrates into biological membranes upon contacting. Preferably, the effector polypeptide has a decreased binding affinity to glycosphingolipids, preferably ganglioside GM2 (CAS No. 19600 Jan. 2) and/or ganglioside GM3 (CAS No. 54827-14-4), and/or to phosphoinositides, preferably phosphatidylinositol 4,5-bisphosphate (PI(4,5)P2,(CAS No. 245126-95-8) and/or phosphatidylinositol (3,4,5)-trisphosphate (PI(3,4,5)P3, compared to the wildtype transmembrane domain of CD95; preferably, said decreased affinity occurs and/or is determinable when the effector polypeptide is integrated in a biological membrane. Preferred means and methods for predicting and/or determining said decreased binding activity are specified herein in the Examples. Also preferably, the effector polypeptide has the activity of reducing pro-tumorigenic signaling in cancer cells, preferably in CD95-positive cancer cells. Also preferably, the effector polypeptide has the activity of inducing apoptosis in a cancer cell or in an activated immune cell, preferably an activated T-cell or an activated peripheral monocyte, or an activated tissue macrophage. Activation markers of T-cells are known to the skilled person and include in particular CD69, CD25, and/or HLA-DR. Methods for measuring induction of apoptosis in cells, in particular cancer cells and activated immune cells, are known in the art and include in particular those described herein in the Examples. Preferably, induction of apoptosis comprises an increase in the fraction of apoptotic cells in a cell population by at least 10%, more preferably at leat 25%, even more preferably at least 50% compared to a control cell population not treated with the effector polypeptide.

In a preferred embodiment, the effector polypeptide further has the activity of causing a change in the lipid composition of host cells. preferably a change in the content of phosphatidylcholines, phosphatidylethanolamines, phosphatidylserines, phosphoinositides, sphingomyelins, ceramides and/or glycosphingolipids. In a further preferred embodiment, the effector polypeptide further has the activity of inducing a cell cycle arrest in a host cell, preferably an arrest in the Gl phase. In a further embodiment, the effector polypeptide further has the activity of decreasing phospho-protein kinase B (pAkt) mediated signaling and/or increasing phospho-epidermal growth factor receptor (pEGFR) mediated signaling. In a further preferred embodiment, the effector polypeptide further has the activity of decreasing the endocytosis rate, which can e.g. be measured as transferrin uptake over time, in a host cell. Furthermore, in a preferred embodiment, the effector polypeptide further has the activity of decreasing cell size, which can e.g. be measured as area of a cell in a microscopic picture thereof, in a host cell. Preferably, said decrease is at least 10%, more preferably at least 25%, even more preferably at least 50%, compared to a control treated host cell. In a further preferred embodiment, the effector polypeptide further has the activity of increasing cell death. Preferably, said increase is at least 30%, more preferably at least 40%, even more preferably at least 60%, compared to a control treated host cell. Furthermore, in a preferred embodiment, the effector polypeptide further has the activity of inducing apoptosis, preferably as specified herein above, more preferably as assessed by caspase-8 cleavage upon EGF exposure.

The term “subject”, as used herein, relates to an animal, preferably a vertebrate, more preferably a mammal, in particular to livestock like cattle, horse, pig, sheep, and goat, to a companion animal such as dog or cat, or to a laboratory animal like a rat, mouse, and guinea pig. Most preferably, the subject is a human. Preferably, the subject is suffering from cancer, from inflammatory disease, and/or neurodegenerative disease, more preferably is in need of treatment and/or prevention of cancer, inflammatory disease, and/or neurodegenerative disease, all as specified elsewhere herein. Preferably, the subject is at increased risk of developing one or more of the aforesaid diseases. More preferably, the subject is known to suffer from and/or is under treatment for one or more of the aforesaid diseases.

The term “cancer”, as used herein, relates to a disease of a subject, characterized by uncontrolled growth by a group of body cells (“cancer cells”). This uncontrolled growth may lead to tumor formation and may be accompanied by intrusion into and destruction of surrounding tissue (invasion) and possibly spread of cancer cells to other locations in the body (metastasis). Preferably, also included by the term cancer is a relapse. Thus, preferably, the cancer is a non-solid cancer, a solid cancer, a metastasis, and/or a relapse thereof. Also preferably, cancer treatment further comprises at least one of surgery, chemotherapy, radiotherapy, and immunotherapy, in particular cell-based immunotherapy. Preferably, the cancer is a CD95-expressing cancer and/or showing a high degree of immune infiltration. Also preferably, the cancer is a cancer having increased RTK activity compared to a control tissue, preferably tissue adjacent to said cancer. More preferably, the cancer is a receptor tyrosine kinase (RTK) and/or integrin dependent cancer, more preferably is an EGFR dependent cancer. Thus, the cancer preferably is a cancer whose cells are inhibited from growing by an RTK inhibitor. Preferably, the RTK is epidermal growth factor receptor (EGFR), thus, the RTK inhibitor preferably comprises, more preferably is, Erlotinib, Afatinib, Dacomitinib, Gefitinib, Lapatinib, Neratinib, Osimertinib, and/or Vandetanib. Preferably, the cancer is brain cancer, more preferably glioblastoma, or pancreatic cancer, preferably pancreatic ductal adenocarcinoma (PDAC).

The term “inflammatory disease” relates to any and all diseases characterized by inappropriate immune activation; thus, the term includes any and all physiological states of a subject in which immune activation causes symptoms of disease, preferably for an extended period of time, more preferably for at least two weeks, still more preferably at least four weeks, even more preferably at least 2 months, most preferably at least six months. Preferably, the inflammatory disease comprises an activation of the immune system to an extent and/or directed to one or more antigens which does not normally occur in a patient in the same physiological situation; thus, inflammatory disease can e.g. be identified by comparing the immune activation in a subject to an immune activation in a reference population of subjects. Appropriate methods, e.g. statistical methods, for such a comparison are known to the skilled person. Preferably, the symptoms caused by the inflammatory disease require medical treatment, more preferably are at least potentially life-threatening, preferably acutely and/or chronically. Thus, the inflammatory disease may in particular be a chronic disease. Preferably, the inflammatory disease comprises inappropriate activation of T-cells. Thus, the inflammatory disease may preferably be Morbus Crohn, Colitis Ulcerosa, Rheumatoid Arthritis, and/or Graft-vs-host disease. The inflammatory disease may, however, also be a remodeling process with an inflammatory component, in particular during and/or after ischemia, in particular after heart ischemia, e.g. in myocardial infarction or in heart insufficiency.

The term “neurodegenerative disease” relates to a disease caused by progressive loss of structure and/or function of neurons in the peripheral and/or central nervous system of a subject. Preferably, the neurodegenerative disease is a neurodegenerative disease of motoneurons and/or a neurodegenerative disease of the central nervous system. Preferably, the neurodegenerative disease is Alzheimer's disease, amyotrophic lateral sclerosis (ALS), Parkinson's disease, or multiple sclerosis. Also preferably, the neurodegenerative disease is a neurodegenerative disease with an inflammatory component, such as Parkinson's disease, or is acute neurodegeneration, in particular after spinal cord injury or stroke.

Advantageously, it was found in the work underlying the present invention that effector polypeptides as specified herein have the property of having a modified lipid interaction, which, in turn, causes modified signaling properties. Notably, it was found that overexpression of an effector polypeptide as claimed in a tumor cell with a wild-type CD95 background is sufficient to outcompete the pro-tumorigenic activities of wildtype CD95 and to induce death of the tumor cell. Thus, outcompeting wildtype lipid-CD95 interaction by an effector polypeptide switches tumorigenic to apoptotic activity.

The definitions made above apply mutatis mutandis to the following. Additional definitions and explanations made further below also apply for all embodiments described in this specification mutatis mutandis.

In view of the above, the present invention also relates to an effector polypeptide comprising the amino acid sequence X¹X²X³LGWLCLLLLPIPLIVX⁴VKX⁵X⁶ (SEQ ID NO:2), wherein X¹ is selected from E, Q, A, N, G, V, L, I, S, T, D, and M, preferably is E or Q; X² is selected from A, G, V, L, and I, X³ is selected from A, G, V, L, I, Q, N, D, E, S, T, M, H, K, and R; and/or X⁴ to X⁶ are independently selected from A, G, V, L, I, Q, N, D, E, S, T, and M. Preferred embodiments of said effector polypeptide have been described herein above. Preferably, the effector polypeptide comprises, preferably consists of, an amino acid selected from the group consisting of SEQ ID NOs: 3 to 4.

The present invention further relates to a polynucleotide comprising a nucleic acid sequence encoding an effector polypeptide according to the present invention.

The term “polynucleotide” is known to the skilled person. As used herein, the term includes nucleic acid molecules comprising or consisting of a nucleic acid sequence or nucleic acid sequences as specified herein. The polynucleotide of the present invention shall be provided, preferably, either as an isolated polynucleotide (i.e. isolated from its natural context) or in genetically modified form. The polynucleotide, preferably, is DNA, including cDNA, or is RNA. The term encompasses single as well as double stranded polynucleotides, as well as circularly closed and linear polynucleotides. Preferably, the polynucleotide is a chimeric molecule, i.e., preferably, comprises at least one nucleic acid sequence, preferably of at least 20 bp, more preferably at least 100 bp, heterologous to the residual nucleic acid sequence(s) or being an artificial nucleic acid sequence. Moreover, preferably, comprised are also chemically modified polynucleotides including naturally occurring modified polynucleotides such as glycosylated or methylated polynucleotides or artificial modified ones such as biotinylated polynucleotides.

As used herein, the term polynucleotide, preferably, includes variants of the specifically indicated polynucleotides. More preferably, the term polynucleotide relates to the specific polynucleotides indicated. It is to be understood, however, that a polypeptide having a specific amino acid sequence may be encoded by a variety of polynucleotides, due to the degeneration of the genetic code. The skilled person knows how to select a polynucleotide encoding a polypeptide having a specific amino acid sequence and also knows, if desired, how to optimize the codons used in the polynucleotide according to the codon usage of the organism used for expressing said polynucleotide. Thus, the term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide, i.e.

shall encode an effector polypeptide. Preferably, said polynucleotide variant is an ortholog, a paralog, or another homolog of the specific polynucleotide. Further variants include polynucleotides comprising nucleic acid sequences which are at least 70%, at least 75%, at least 80%, at least 85%, at least 90%, at least 95%, at least 98% or at least 99% identical to the specifically indicated nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated as specified herein above.

A polynucleotide comprising a fragment of any of the specifically indicated nucleic acid sequences is also encompassed as a variant polynucleotide of the present invention. The fragment shall still encode an effector polypeptide as specified herein above which still has the activity as specified. Accordingly, the effector polypeptide encoded may comprise or consist of the domains of the effector polypeptide of the present invention conferring said biological activity. A fragment as meant herein, preferably, comprises at least 69, more preferably at least 100, still more preferably at least 250, most preferably at least 500, consecutive nucleotides of any one of the specific nucleic acid sequences or encodes an amino acid sequence comprising at least 23, preferably at least 50, more preferably at least 100, still more preferably at least 150, most preferably at least 200 consecutive amino acids of any one of the specific amino acid sequences. Preferably, the polynucleotide comprises a nucleic acid sequence selected from the group consisting of SEQ ID NOs: 16, 18, 21, 24, and 27.

The polynucleotides of the present invention either consist, essentially consist of, or comprise the aforementioned nucleic acid sequences. Thus, they may contain further nucleic acid sequences as well. Specifically, the polynucleotides of the present invention may encode fusion polypeptides wherein one partner of the fusion polypeptide is an effector polypeptide being encoded by a nucleic acid sequence recited above. Such fusion proteins may comprise as additional part further polypeptides as specified herein above, such as tags and linkers

Preferably, the polynucleotide comprising a nucleic acid sequence encoding an effector polypeptide is comprised in an expression construct allowing for expression of said polynucleotide in a host cell. The term “expression construct”, as used herein, refers to a heterologous polynucleotide comprising the aforementioned polynucleotide encoding the effector polypeptide as well as nucleic acid sequences required for expression of the polynucleotide encoding the effector polypeptide. Typically, such additional nucleic acid sequences, which preferably are heterologous to the polynucleotide encoding the effector polypeptide, may be promoter sequences, regulatory sequences and/or transcription termination sequences, such as terminators. Preferably, the expression construct is a eukaryotic expression construct, i.e. an expression construct comprising all elements required for expression, preferably inducible expression, in a eukaryotic host cell. Suitable expression control sequences are well known in the art and include in particular the CMV promoter.

Preferably, the polynucleotide comprising a nucleic acid sequence encoding an effector polypeptide, more preferably the expression construct comprising the polynucleotide comprising a nucleic acid sequence encoding an effector polypeptide, is comprised in a vector.

The term “vector”, as used herein, relates to any polynucleotide adapted for stably maintaining the polynucleotide and/or the expression construct as specified herein above in a host cell. Thus, the term vector preferably encompasses phage, plasmid, and viral vectors as well artificial chromosomes, such as bacterial or yeast artificial chromosomes. Preferably, the vector is a plasmid or a virus-derived vector, preferably a replication-incompetent viral vector. Moreover, the term also relates to targeting constructs which allow for random or site-directed integration of the targeting construct into genomic DNA of a host cell. Such target constructs, preferably, comprise DNA of sufficient length for either homologous or heterologous recombination. The vector encompassing the polynucleotide and/or the expression construct as specified herein above, preferably, further comprises at least one selectable marker for propagation and/or selection of a host cell. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Preferably, the vector is a vertebrate vector, more preferably a mammalian vector, or a shuttle vector. Preferably, the vector is an expression vector and/or a gene transfer or targeting vector. Methods which are well known to those skilled in the art can be used to construct recombinant polynucleotides and vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Preferably, the vector is an AAV vector, preferably comprising a nucleic acid sequence selected from the group consisting of SEQ ID NOs: 17, 19, 22, 25, and 28. Also preferably, the vector is a lentiviral vector, preferably comprising a nucleic acid sequence selected from the group consisting of SEQ ID NOs: 20, 23, 26, and 29.

The present invention also relates to a host cell comprising the effector polypeptide according to the present invention and/or the polynucleotide according to the present invention.

As used herein, the term “host cell” relates to any cell capable of receiving, and preferably maintaining, the polynucleotide and/or the effector polypeptide, as specified herein above. Preferably, the host cell is capable of expressing an effector polypeptide encoded on the expression polynucleotide and/or expression vector as specified herein above. Also preferably, the host cell is a cell comprising at least one biological membrane, more preferably a biological membrane into which the effector polypeptide integrates or is integrated as specified herein above. Preferably, the host cell is a eukaryotic cell, preferably an animal cell, e.g. an insect cell or a mammalian cell. More preferably, the host cell is a cell of a livestock, companion, or laboratory animal. Most preferably, the host cell is a human cell. Preferably, the host cell is a cell expressing CD95. Preferably, the host cell is a cancer cell, an immune cell, preferably an activated immune cell, preferably an activated T cell, an activated monocyte, or an activated tissue macrophage, or is a neural cell, preferably an astrocyte or a neural stem cell. Preferably, the host cell is a cancer cell, an immune cell, or a neural cell expressing CD95. The host cell may, however, also be a host cell producing viral particles and/or liposomes comprising the effector polypeptide as specified herein and/or the polynucleotide as specified herein. The host cell may be of a cell type causing or contributing to disease, in which case it may also be referred to as “disease-mediating cell”; thus, disease-mediating cells may in particular be cancer cells in cancer, and activated immune cells as specified herein above in inflammatory disease and/or acute or chronic neurodegenerative disease.

The present invention further relates to a pharmaceutical composition comprising (A) an active agent selected from the list consisting of an effector polypeptide according to the present invention, a polynucleotide according to the present invention, and/or a host cell according to the present invention and (B) an excipient.

The terms “medicament” and “pharmaceutical composition” are used essentially interchangeably herein and are, in principle, known to the skilled person. As referred to herein, the terms relate to any composition of matter comprising the specified active agent(s) as pharmaceutically active compound(s) and one or more excipient. The pharmaceutically active compound(s) can be present in liquid or dry, e.g. lyophilized, form. For example, the pharmaceutically active compound can be present together with glycerol and/or stabilizers (e.g., reducing agents, human serum albumin). The medicament is, typically, administered systemically or topically, preferably orally, by inhalation, or parenterally, e.g. by intravenous administration; in case of cancer treatment, topical administration may be intratumoral or peritumoral, and/or topical at a site of tumor excision. Administration may, however, also be into a blood vessel, typically an artery, afferent to an intended site of effect, such as a tumor. However, depending on the nature of the formulation and the desired therapeutic application, the medicament may be administered by other routes as well. The pharmaceutically active compound is the active agent or drug of the medicament, and is preferably administered in conventional dosage forms prepared by combining the drug with standard pharmaceutical carriers according to conventional procedures. These procedures may involve mixing, granulating, and compression, or dissolving the ingredients as appropriate to obtain the desired preparation.

It will be appreciated that the form and character of the pharmaceutical acceptable excipient, e.g. carrier or diluent, is dictated by the amount of active ingredient with which it is to be combined, the route of administration, and other well-known variables. The excipient(s) must be acceptable in the sense of being compatible with the other ingredients of the formulation and being not deleterious to the recipient thereof. The excipient employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, physiological saline, Ringer's solutions, dextrose solution, and Hank's solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The excipient(s) is/are selected so as not to affect the biological activity of the combination. The excipient may, however, also be selected to improve uptake of the active agent into a target cell, in particular the host cell. Thus, the excipient may also be a viral particle and/or a lipid vesicle, preferably a viral particle and/or a lipid vesicle known to mediate entry and/or fuse with the target cell of interest. In accordance, preferably, the pharmaceutical composition comprises a polynucleotide as specified herein, more preferably an expression construct as specified herein, most preferably an expression vector as specified herein, comprised in a viral particle, preferably an adeno associated virus particle, a retrovirus particle, or an adenovirus particle.

A therapeutically effective dose refers to an amount of the effector polypeptide or expression construct encoding the same to be used in a medicament which prevents, ameliorates or cures the symptoms accompanying a disease or condition referred to in this specification. Therapeutic efficacy and toxicity of a drug can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and by clinical factors. As is well known in the medical arts, dosages for any one patient depends upon many factors, including the patient's size, age, the particular formulation of the medicament to be administered, sex, time and route of administration, general health, and other drugs being administered concurrently. The medicament referred to herein is, preferably, administered at least once, e.g. as a bolus. However, the medicament may be administered more than one time and, preferably, at least twice, e.g. permanently or periodically after defined time windows.

Progress can be monitored by periodic assessment. Dosage recommendations may be indicated in the prescriber or user instructions in order to anticipate dose adjustments depending on the considered recipient.

The medicament according to the present invention may comprise further active agents in addition to the aforementioned active agent(s). Preferably, the pharmaceutically active compound according to the invention is to be applied together with at least one further drug and, thus, may be formulated together with this at least one further drug as a medicament. More preferably, in case of cancer treatment, said at least one further active agent is a chemotherapeutic agent or an immunotherapeutic agent; in case of inflammatory disease treatment, said at least one further active agent is an anti-inflammatory agent; and in case of treatment of neurodegenerative disease, said at least one further active agent is a neurostimulatory drug. The medicament may further comprise a CD95 agonist, such as a trimeric CD95L or an artificial mimic thereof. Also, it is to be understood that the formulation of a pharmaceutical composition preferably takes place under GMP standardized conditions or the like in order to ensure quality, pharmaceutical safety, and effectiveness of the medicament.

The present invention also relates to an effector polypeptide according to the present invention, a polynucleotide according to the present invention, a host cell according to the present invention, and/or a pharmaceutical composition according to the present invention, for use in medicine.

The present invention further relates to an effector polypeptide according to the present invention, a polynucleotide according to the present invention, a host cell according to the present invention, and/or a pharmaceutical composition according to the present invention, for use in treating and/or preventing cancer, inflammatory disease, and/or acute or chronic neurodegenerative disease.

The terms “cancer”, “inflammatory disease”, and “neurodegenerative disease” have been specified herein above.

The terms “treating” and “treatment” refer to an amelioration of the diseases or disorders referred to herein or the symptoms accompanied therewith. Said treating as used herein includes an entire restoration of health with respect to the diseases or disorders referred to herein, as well as preventing the worsening of the disease or disorders or said at least one symptom. Thus, if progression of disease or aggravation of at least one symptom associated therewith is prevented, or if there is amelioration or cure of the disease or at least a symptom associated therewith, the treatment shall be deemed to be effective. It will be understood that treating may not be effective in all subjects. However, according to the present invention it is envisaged that treatment will preferably be effective in at least a statistically significant portion of subjects to be treated. It is well known to the skilled artisan how to determine a statistically significant portion of subjects that can be effectively treated. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the finding of effective treatment will be correct for at least 60%, at least 70%, at least 80%, or at least 90% of the subjects of a given cohort or population. Preferably, effectiveness of treatment is determined by determining survival of treated subjects of a given cohort or population, preferably determining a disease-free survival rate, a progression-free survival rate, and/or an overall survival rate. However, other parameters, such as increase in survival time compared to a control cohort, may be determined.

Preferably, treatment comprises additional treatment steps, e.g. those specified elsewhere herein, and in particular co-administration of a CD95 agonist, such as a trimeric CD95L or an artificial mimic thereof. Also preferably, treatment further comprises administration of at least one inhibitor of a receptor tyrosine kinase (RTKs). RTKs are known in the art; preferably, the RTK is a member of the epidermal growth factor receptor (EGFR) family, more preferably is EGFR, most preferably is human EGFR, also known as Herl, e.g. Genbank Acc No. NP_005219.2 or an alternative isoform thereof. Thus, the inhibitor of a receptor tyrosine kinase comprises, preferably is, an EGFR inhibitor, preferably is Erlotinib (CAS NO. 183321-74-6), Afatinib (CAS NO. 850140-72-6), Dacomitinib (CAS No. 1110813-31-4), Gefitinib (184475-35-2), Lapatinib (CAS No. 231277-92-2), Neratinib (CAS No. 698387 Sep. 6), Osimertinib (CAS No. 1421373-65-0), and/or Vandetanib (CAS No. 443913-73-3).

Preferably, treating cancer is reducing tumor burden in a subject and/or preventing metastasis. As will be understood by the skilled person, effectiveness of treatment of e.g. cancer is dependent on a variety of factors including, e.g. cancer stage and cancer type. Preferably, treating causes cancer cells to enter apoptosis. Thus, preferably, treating has the effect of causing a tumor to stop growing, more preferably to cause regression of a tumor, more preferably of causing a tumor to resolve. As specified herein elsewhere, cancer treatment may comprise additional treatments, such as surgery, radiotherapy, chemotherapy, immunotherapy, and the like.

Treating inflammatory disease, preferably, comprises ameliorating inflammation in a subject or in affected tissues and/or organs thereof, and/or of symptoms associated with said inflammation. Treatment of inflammation may also comprise reducing immune response in a subject or in affected tissues and/or organs thereof to normal levels. Treatment may, in particular in case of chronic inflammatory disease, comprise preventing progress of disease and/or aggravation of at least one symptom associated therewith.

Treating neurodegenerative disease, preferably, comprises slowing or preventing loss of neuronal cells and/or function thereof in a subject and/or slowing, preventing, or reversing aggravation of symptoms associated with said neurodegeneration. Preferably, treatment of neurodegenerative disease comprises preventing progress of disease and/or aggravation of at least one symptom associated therewith in chronic degenerative disease.

The term “preventing” as used herein refers to avoiding the onset of the disease or at least one symptom associated therewith. The prevention as referred to herein can be typically achieved either for the period during which a drug is administered. If the administration of the drug is stopped, however, the prevention may not persist for an unlimited time but may remain present for a certain preventive time window after application of the drug. Typically, a preventive time window in accordance with the present invention may be at least 1 day, at least 7 days, at least 2 weeks, at least 4 weeks, at least 6 months, or at least a year. However, the preventive time window may also depend on the dosage of a drug as well as the mode of administration or the kind of formulation. For example, if a high dosage is applied, usually longer preventive time windows can be achieved. The same holds true if slow release formulations of a drug are administered or the drug is administered via routes that do not lead to immediate metabolization of a drug in the subject. In such cases, the preventive time window may be increased up to several weeks, months or even years. It will be understood that prevention might not be effective in all subjects. However, according to the present invention it is envisaged that prevention preferably will be effective in at least a statistically significant portion of subjects. It is well known to the skilled artisan how to determine a statistically significant portion of subjects that can be effectively prevented. Whether a portion is statistically significant can be determined without further ado by the person skilled in the art using various well known statistic evaluation tools as discussed above. Prevention of disease may in particular be appropriate in a subject having a disposition for acquiring said disease, e.g. a genetic disposition or a lifestyle-related disposition.

Moreover, the present invention relates to a method of treating and/or preventing cancer, inflammatory disease, or acute or chronic neurodegenerative disease in a subject in need of such treatment comprising (a) contacting said subject with an effector polypeptide according to the present invention, a polynucleotide according to the present invention, a host cell according to the present invention, and/or a pharmaceutical composition according to the present invention; and (b) thereby treating cancer, inflammatory disease, or acute or chronic neurodegenerative disease.

The method of the present invention may comprise steps in addition to those explicitly mentioned above. For example, further steps may relate, e.g., to diagnosing or determining a risk for acquiring cancer, inflammatory disease, or acute or chronic neurodegenerative disease before step a), and/or additional treatments, preferably as specified elsewhere herein, before, during, or after step a). Moreover, one or more of said steps may be assisted or performed by automated equipment.

The present invention also relates to a kit comprising an effector polypeptide according to the present invention, a polynucleotide according to the present invention, a host cell according to the present invention, and/or a pharmaceutical composition according to the present invention, in a housing.

The term “kit”, as used herein, refers to a collection of the aforementioned compounds, means or reagents. The components of the kit may be comprised by separate vials (i.e. as a kit of separate parts) or provided in a single vial, e.g. as a composition as specified herein above. The housing of the kit preferably allows translocation of the compounds of the kit, in particular common translocation; thus, the housing may in particular be a transportable container comprising all specified components. Moreover, it is to be understood that the kit of the present invention may be used for practicing at least one of the methods referred to herein above. It is envisaged that all components may be provided in a ready-to-use manner for practicing a method referred to above. Further, the kit preferably contains instructions for carrying out said methods, e.g. dosage instructions. The instructions can be provided by a user manual in paper- or electronic form. The kit preferably comprises further components, preferably at least one further therapeutic agent as specified herein above, an excipient, and/or a means of administration, in particular a syringe and/or a needle, and/or an IV infusion equipment.

The present invention also relates to a use, preferably an in vitro use, of an effector polypeptide according to the present invention, a polynucleotide according to the present invention, a host cell according to the present invention, and/or a pharmaceutical composition according to the present invention for inducing apoptosis in a host cell.

In a preferred embodiment, the present invention further relates to a use, preferably an in vitro use, of the effector polypeptide for causing a change in the lipid composition of a host cell, for inducing a cell cycle arrest in a host cell, for decreasing phospho-protein kinase B (pAkt) mediated signaling and/or increasing phospho-epidermal growth factor receptor (pEGFR) mediated signaling in a host cell, for decreasing the endocytosis rate in a host cell, for decreasing cell size of a host cell, and/or for increasing cell death, preferably apoptosis, in particular upon EGF stimulation, all as specified herein above and in the Examples.

In a preferred embodiment, the term “change in lipid composition”, as used herein, relates to a, preferably significant, change in the relative fractions of lipid classes in a cell, preferably in the plasma membrane of a cell. Preferably, a change in the lipid composition comprises a change in at least one lipid class selected from the list consisting of phosphatidylcholines, phosphatidylethanolamines, phosphatidylserines, phosphoinositides, sphingomyelins, ceramides and/or glycosphingolipids. Methods for determining lipid composition in cells and in particular cytoplasmic membranes are known to the skilled person.

The term “cell cycle arrest”, in a preferred embodiment, is known to the skilled person and more preferably relates to an arrest in the G1 phase of the cells cycle. Preferably, after inducing a cell cycle arrest, in particular by contacting a host cell with an effector polypeptide as specified herein, at least 50%, more preferably at least 70%, even more preferably at least 75% of the host cells in a population are in Gl phase. Preferably, cell cyle is determined after of from 3days to 30 days, preferably 1 to 2 weeks, after inducing a cell cycle arrest, e.g. by transduction of said host cells with a viral expression vector causing expression of at least one effector polypeptide as specified herein. Methods for determining cell cycle in cells are known to the skilled person.

In a preferred embodiment, the terms “pAkt signaling” and “pEGFR signaling” are known to the skilled person to relate to well-known signaling pathways in particular in mammalian cells. Activity of said signaling pathways is preferably determined by determining pAkt and/or pEGFR, respectively, e.g. by immunological methods known in the art.

The term “endocytosis rate”, in a preferred embodiment, is known to the skilled person to relate to the rate of uptake of extracellular components into cells, preferably via clathrin coated pits. Methods for measuring the endocytosis rate are known in the art, e.g. the transferrin uptake assay, as also described in the Examples.

The term “decreasing cell size”, in a preferred embodiment, is understood by the skilled person and appropriate method of determining cell size are known in the art, e.g. via microscopy, PACS, and the like. Preferably, decreasing cell size is decreasing cell size by at least 25%, more preferably at least 40%, even more preferably at least 50%, compared to an untreated control.

In a preferred embodiment, the term “cell death” is understood by the skilled person and preferably relates to each and every process in which a host cell ceases to carry out its functions, preferably ceases to metabolize, in particular ceases to metabolize glucose and/or to synthesize proteins. Cell death may be accompanied by structural and morphological changes in the host cell, in particular cell lysis. Cell death may, however, also occur in the absence of discernible structural and/or morhological changes. Methods for determining cell death are known in the art and include in particular trypan blue staining of cells, preferably as described herein in the Examples. The term cell death in particular includes apoptosis, autophagy, other forms of programmed cell death, necrosis, and senescence. The terms “apoptosis”, “autophagy”, “programmed cell death”, “necrosis”, and “senescence” are known to the skilled person, e.g. from standard textbooks.

In view of the above, in a preferred embodiment, the present invention further relates to a method for causing a change in the lipid composition of a host cell, for inducing a cell cycle arrest in a host cell, for decreasing phospho-protein kinase B (pAkt) mediated signaling and/or increasing phospho-epidermal growth factor receptor (pEGFR) mediated signaling in a host cell, for decreasing the endocytosis rate in a host cell, for decreasing cell size of a host cell, and/or for increasing cell death, in particular for increasing apoptosis upon EGF exposure, the method comprising administration of an effector polypeptide as specified herein. Said method may be an in vitro method, e.g. performed on cultured cells. The method may also be performed on a non-human experimental animal, in which case said experimental animal preferably is sacrificed after said administration. The method may, however, also be comprised in a method of treatment as specified elsewhere herein; in such case, the subject treated preferably is a mammal, more preferably a human, and/or the host cell is a cancer cell or an immune cell, as specified herein above.

Further, the present invention relates to a method for identifying an inhibitor of an interaction of CD95 with at least one membrane lipid (interaction inhibitor), comprising
(a) contacting a host cell comprising CD95 or a fragment thereof and at least one membrane lipid with a candidate inhibitor;
(b) determining interaction between CD95 or said fragment thereof and membrane lipids;
(c) comparing the interaction determined in step (b) to a control interaction; and,
(d) based on the comparison of step (d), identifying an interaction inhibitor.

The method for identifying an inhibitor of an interaction of CD95, preferably, is an in vitro method. In case the method is performed in an experimental animal, the method preferably comprises step (e) sacrificing the experimental animal. Moreover, the method may comprise steps in addition to those specifically mentioned and may be assisted or performed by automated equipment.

The term “membrane lipid”, as used herein, relates to any lipid present in a membrane of a host cell, preferably a disease-mediating cell, as specified herein above. Preferably, said membrane is a cytoplasmic membrane or a membrane of an endosome or of an endolysosome, more preferably is a cytoplasmic membrane. Preferably, the membrane lipid is a ganglioside, preferably ganglioside GM2 or ganglioside GM3; or is a phosphoinositide, preferably PI(4,5)PI₂ (phosphatidylinositol 4,5-bisphosphate) or PI(3,4,5)PI₃ (phosphatidylinositol 3,4,5-trisphosphate).

The term “candidate inhibitor” is used herein in a broad sense relating to any chemical compound suspected to inhibit an interaction of CD95 with at least one membrane lipid; thus, the term, preferably, relates to any compound not having previously been tested negative for inhibiting an interaction of CD95 with at least one membrane lipid. Preferably, a candidate inhibitor is identified as an interaction inhibitor in case the interaction between said membrane lipid and said CD95 or fragment thereof, or a corresponding readout, is modulated, preferably decreased, by at least 10%, more preferably at least 20%, most preferably at least 50%, compared to the control interaction.

Determining interaction between CD95 or a fragment thereof and membrane lipids may be accomplished by any method for determining molecular interaction known to and deemed appropriate by the skilled person. Preferably, the determining method is indirect, i.e. determines a physiological outcome of said interaction in the cell, e.g. apoptosis induction. More preferably, the method is a direct method of determining molecular interaction. Corresponding methods are known to the skilled person and include in particular optical methods, e.g. FRET methods as known e.g. from Nakano et al. (2021), BBA Biomembranes 1863:186323 and/or microscopic methods of determining interaction; in such case, preferably, the CD95 or fragment thereof is labeled with a first detector compound, preferably a first fluorescent label; and/or the at least one membrane lipid is labeled with a second detector compound, preferably a second fluorescent label. The term “detector compound”, as used herein, relates to a compound adapted for making the presence of a molecule or complex comprising said detector compound detectable. Typically, the detector compound has a detectable property, typically an optical or/and enzymatic property. It is, however, also envisaged that said detectable property is the property of emitting radioactivity. Also, systems in which a first and a second detector compound together form an enzymatically active polypeptide are known in the art. Preferably, the first and the second detector compounds are detectable by optical means, more preferable are dyes, most preferably are fluorescent labels. As is understood by the skilled person, in FRET application, the first and the second detector compounds are preferably fluorescent labels with appropriately overlapping emission/absorption spectra. It is, however, also envisaged that interaction is determined by other methods, e.g. using photoactivatable cross-linking groups attached to CD95 or a fragment thereof and/or to a membrane lipid; appropriate methods are known to the skilled person and are reviewed e.g. in Haberkant & Holthuis (2014), BBA-Mol Cell Biol Lipids 1841 (8): 1022.

According to step (c) the interaction determined in step (b) is compared to a control interaction. Said control interaction is determined in a host cell not contacted with the candidate inhibitor and/or in a host cell comprising the effector polypeptide as specified herein above.

The present invention also relates to an inhibitor of interaction of CD95 with at least one membrane lipid for use in medicine, preferably for use in treating cancer, inflammatory disease, or acute or chronic neurodegenerative disease.

As specified herein above, an “inhibitor of interaction of CD95 with at least one membrane lipid”, also referred to as “interaction inhibitor”, is a chemical compound inhibiting interaction of a membrane lipid and a CD95 or fragment thereof or a corresponding readout by at least 10%, more preferably at least 20%, most preferably at least 50%, compared to the control interaction. Preferably, the interaction inhibitor is a direct interaction inhibitor, i.e. a chemical compound binding to the transmembrane domain of CD95, preferably SEQ ID NO:1, or to a membrane lipid or part thereof, e.g. the headgroup of the membrane lipid, thereby preventing CD95 or a fragment thereof and the membrane lipid from interacting. Thus, the direct interaction inhibitor preferably is an effector polypeptide as specified herein above. The direct interaction inhibitor may, however, also be a small molecule compound, preferably with a molecular mass of less than 2 kDa, more preferably less than 1 kDa, an antibody or fragment thereof, such as a nanobody, an aptamer, or the like. The small molecule inhibitor may in particular be a lipid, e.g. GB3, or a, preferably soluble, derivative thereof, having the activity of inhibiting interaction of CD95 with at least one membrane lipid; thus, the small molecule inhibitor may in particular be a soluble mimick of GB3 having the activity of inhibiting interaction of CD95 with at least one membrane lipid. The direct inhibitor may, however, also be a binding agent binding to the headgroup of a membrane lipid, in particular GM2 and/or GM3, and having the activity of inhibiting interaction of CD95 with at least one membrane lipid. Preferably, said binding agent is soluble, i.e. is soluble at a concentration of at least 1 mg/ml in an aqueous solution, preferably under standard conditions. Thus, the binding agent binding to the headgroup of a membrane lipid preferably is a lectin, an antibody, an aptamer, or fragment of any of the aforesaid having the specified activity.

The interaction inhibitor may, however, also be an indirect interaction inhibitor, i.e. a compound, modulating, preferably decreasing, the concentration of a membrane lipid as specified above in a host cell. As the skilled person understands, interaction inhibition may be accomplished by decreasing the concentration of at least one of the interaction partners, preferably GM2, but may also be accomplished by increasing the concentration of a competing antagonistic compound; a competing antagonistic compound preferably is a lipid, e.g. GB3 or a, preferably soluble, structural analog thereof, having the activity of decreasing concentration of a membrane lipid interacting with CD95 in a biological membrane by decreasing the concentration of said interacting membrane lipid. Biochemical pathways of e.g. ganglioside biosynthesis are known e.g. from Yu et al. (2011), J Oleo Sci 60 (10): 537), as are inhibitors of respective enzymes (cf. e.g. WO 01/39804); thus, the interaction inhibitor preferably is selected from the group consisting of tunicamycin (CAS NO. 11089-65-9), gangliosides GQ1b, GT1a, and GT1b. Also, the indirect interaction inhibitor may be a high-molecular weight compound binding to at least one membrane lipid biosynthetic enzyme, preferably as specified herein below, such that its activity is inhibited; appropriate compounds are known in the art and include e.g. antibodies, aptamers, DARPINs, and the like. Preferably, the indirect interaction inhibitor is an RNAi agent, at least one gRNA, or a ribozyme, reducing expression of at least one membrane lipid biosynthetic enzyme, preferably a ganglioside biosynthetic enzyme, to an extent causing the concentration of ganglioside GM2 and/or ganglioside GM2 in a membrane extract of a host cell to decrease by at least 10%, preferably at least 20%, more preferably at least 50% compared to a control cell not treated with said indirect interaction inhibitor. Preferably, the gene expression of which is inhibited is GM3 synthase, preferably human GM3 synthase (CMP-sialic acid: lactosylceramide α2-3 sialyltransferase, also known as ST-I) or GM2 synthase, preferably human GM2 synthase (UDP-N-Acetylgalactosamine: GM3 B1-4 Nacetylgalactosaminyltransferase).

As used herein, the term “RNAi agent” refers to an shRNA, an siRNA agent, or an miRNA agent as specified below, causing expression of at least one membrane lipid biosynthetic enzyme in a host cell to decrease compared to a control host cell. The RNAi agent is of sufficient length and complementarity to stably interact with the target RNA, i.e. it comprises at least 15, at least 17, at least 19, at least 21, at least 22 nucleotides complementary to the target RNA. By “stably interact” is meant interaction of the RNAi agent or its products produced by the cell with a target RNA, e.g., by forming hydrogen bonds with complementary nucleotides in the target RNA under physiological conditions. As the skilled person understands, the RNAi agent may also be a chemical derivative of a polynucleotide, e.g. a morpholino.

The term “siRNA agent” as meant herein encompasses: a) a dsRNA consisting of at least 15, at least 17, at least 19, at least 21 consecutive nucleotides base-paired, i.e. forming hydrogen bonds with complementary nucleotides. b) a small interfering RNA (siRNA) molecule or a molecule comprising an siRNA molecule. The siRNA is a single-stranded RNA molecule with a length, preferably, greater than or equal to 15 nucleotides and, preferably, a length of 15 to 49 nucleotides, more preferably 17 to 30 nucleotides, and most preferably 17, 18, 19, 20, 21, 22, 23, 24, 25, 26, 27, 28, 29, or 30 nucleotides. According to the present invention, the term “molecule comprising a siRNA molecule” includes RNA molecules from which a siRNA is processed by a cell, preferably by a mammalian cell. Thus, a molecule comprising a siRNA molecule, preferably, is a small hairpin RNA, also known as shRNA. As used herein, the term “shRNA” relates to a, preferably artificial, RNA molecule forming a stem-loop structure comprising at least 10, preferably at least 15, more preferably at least 17, most preferably at least 20 nucleotides base-paired to a complementary sequence on the same mRNA molecule (“stem”), i.e. as a dsRNA, separated by a stretch of non-base-paired nucleotides (“loop”). c) a polynucleotide encoding a) or b), wherein, preferably, said polynucleotide is operatively linked to an expression control sequence.

It is, however, also contemplated that the RNAi agent is a miRNA agent. A “miRNA agent” as meant herein encompasses: a) a pre-microRNA, i.e. a mRNA comprising at least 30, at least 40, at least 50, at least 60, at least 70 nucleotides base-paired to a complementary sequence on the same mRNA molecule (“stem”), i.e. as a dsRNA, separated by a stretch of non-base-paired nucleotides (“loop”). b) a pre-microRNA, i.e. a dsRNA molecule comprising a stretch of at least 19, at least 20, at least 21, at least 22, at least 23, at least 24, at least 25 base-paired nucleotides formed by nucleotides of the same RNA molecule (stem), separated by a loop. c) a microRNA (miRNA), i.e. a dsRNA comprising at least 15, at least 17, at least 18, at least 19, at least 21 nucleotides on two separate RNA strands. d) a polynucleotide encoding a) or b), wherein, preferably, said polynucleotide is operatively linked to an expression control sequence. As will be appreciated, the miRNA agent may be a naturally occurring miRNA; more preferably, the miRNA agent is an artificial miRNA.

The term “ribozyme”, as used herein, refers to catalytic RNA molecules possessing a well-defined tertiary structure that allows for catalyzing either the hydrolysis of one of their own phosphodiester bonds (self-cleaving ribozymes), or the hydrolysis of bonds in other RNAs, but they have also been found to catalyze the aminotransferase activity of the ribosome. The ribozymes envisaged in accordance with the present invention are, preferably, those which specifically hydrolyze the target RNAs. In particular, hammerhead ribozymes are preferred in accordance with the present invention. How to generate and use such ribozymes is well known in the art (see, e.g., Hean & Weinberg (2008), RNA and the Regulation of Gene Expression: A Hidden Layer of Complexity, Chapter 1. Caister Academic Press).

Also preferably, the indirect interaction inhibitor comprises at least one, preferably two, gRNAs, i.e. preferably CRISPR/Cas targeting oligonucleotides, targeting a gene as specified herein above. The CRISPR/Cas system has been known for several years as a convenient system for inducing knock-out mutations, i.e. deletions, preferably of chromosomal genes. The skilled person knows how to design appropriate oligonucleotides, which are, preferably, expressed from a vector, to induce deletion of a DNA sequence of interest. Preferably, said deletion is a partial deletion, more preferably deletion of a portion of the gene essential for function; most preferably said deletion is a complete deletion of at least the whole coding region.

The present invention also relates to a method of identifying a subject suffering from cancer, inflammatory disease, or acute or chronic neurodegenerative disease to be susceptible to treatment with an inhibitor of interaction of CD95 with at least one membrane lipid, said method comprising
(A) determining CD95 expression and/or dependency from receptor tyrosine kinase activity of disease-mediating cells of said subject, and
(B) identifying a subject susceptible to treatment with an inhibitor of interaction of CD95 with at least one membrane lipid based on the determination of step (A).

The method of identifying a subject susceptible to treatment with an interaction inhibitor, preferably, is an in vitro method. Moreover, the method may comprise steps in addition to those specifically mentioned and may be assisted or performed by automated equipment. Preferably, in the method, a subject susceptible to treatment with an inhibitor of interaction of CD95 with at least one membrane lipid is identified if said disease-mediating cells are found to express CD95 and/or to be dependent on receptor tyrosine kinase activity in step (A).

The term “disease-mediating cell” has been specified herein above. Methods of determining CD95 expression are known in the art and include in particular methods of detecting transcripts encoding CD95, e.g. PCR methods, and/or immunological methods, such as immunoblot, ELISA, and the like. CD95 expression may, however, also be determined by other methods, e.g. by MS analysis of peptides derived from a disease-mediating cell.

Methods for determining dependency from receptor tyrosine kinase activity are also known in the art. Preferably, said method comprise administering an RTK inhibitor, preferably an EGFR inhibitor, to disease-mediating cells and determining growth of the treated cells compared to control (untreated) cells. Disease-mediating cells showing at least 10%, preferably at least 20%, more preferably at least 50% growth reduction, as determined e.g. as cell count, compared to a control population, preferably are identified to be dependent from receptor tyrosine kinase activity. Dependency from RTK activity may, however, also be determined by determining levels of intracellular tyrosine phosphorylation, e.g. using a generic anti-phosphotyrosine antibody, which may be applied in immunoblots, in immunohistochemistry applications, or on a single-cell level e.g. via FACS (Balta et al. (2019), Cell Reports 29 (8): 2295).

The present invention also relates to a method of treating a subject suffering from cancer, inflammatory disease, or acute or chronic neurodegenerative disease with an inhibitor of interaction of CD95 with at least one membrane lipid, said method comprising steps (A) and (B) of the method of identifying a subject susceptible to treatment with an interaction inhibitor, and further step
(C) treating a subject identified to be susceptible to treatment by an inhibitor of interaction of CD95 with at least one membrane lipid by administering an inhibitor of interaction of CD95 with at least one membrane lipid.

The aforesaid treating method, preferably, is at least partially an in vivo method. Moreover, the method may comprise steps in addition to those specifically mentioned and may be assisted or performed by automated equipment. Interaction inhibitors to be used in said treatment are those described herein above and/or those identified by the method for identifying an inhibitor of an interaction of CD95 with at least one membrane lipid, also as specified herein above.

In view of the above, the following embodiments are particularly envisaged:

### EMBODIMENT 1

An Effector Polypeptide Comprising
(i) an amino acid sequence at least 70% identical to the amino acid sequence RSNLGWLCLLLLPIPLIVWVKRK (SEQ ID NO:1); and
(ii) an exchange of an amino acid to a non-identical amino acid at at least one position selected from the list consisting of positions 1, 2, 3, 19, 22 and 23 of the amino acid sequence of (i).

### EMBODIMENT 2

The effector polypeptide of embodiment 1, wherein said amino acid exchange(s) is/are selected from the list consisting of R1Q, R1E, R1A, S2A, N3A, R1E/N3A, R1A/N3A, W19A, R22A, R22E, K23A, K23E, R22A/K23A, R22E/K23E, W19A/R22A/K23A, W19A/R22E/K23E, and any combination thereof.

### EMBODIMENT 3

The effector polypeptide of embodiment 1 or 2, wherein said amino acid exchange is R1Q, R1E, or R1A.

### EMBODIMENT 4

The effector polypeptide of any one of embodiments 1 to 3 comprising at least two, in an embodiment at least three amino acid exchanges.

### EMBODIMENT 5

The effector polypeptide of any one of embodiments 1 to 4 wherein said amino acid exchange(s) is/are selected from the list consisting R1E, R22E, and K23E.

### EMBODIMENT 6

The effector polypeptide of any one of embodiments 1 to 5, wherein said amino acid exchange is R1Q.

### EMBODIMENT 7

The effector polypeptide of any one of embodiments 1 to 6, wherein said effector polypeptide comprises amino acid sequence the X¹X²X³LGWLCLLLLPIPLIVX⁴VKX⁵X⁶ (SEQ ID NO:2), wherein
X¹ is selected from E, Q, A, N, G, V, L, I, S, T, D, and M, preferably is E or Q;
X² is selected from A, G, V, L, and I,
X³ is selected from A, G, V, L, I, Q, N, D, E, S, T, M, H, K, and R; and/or
X⁴ to X⁶ are independently selected from A, G, V, L, I, Q, N, D, E, S, T, and M.

### EMBODIMENT 8

An effector polypeptide comprising the amino acid sequence X¹X²X³LGWLCLLLLPIPLIVX⁴VKX⁵X⁶ (SEQ ID NO:2), wherein
X¹ is selected from E, Q, A, N, G, V, L, I, S, T, D, and M, preferably is E or Q;
X² is selected from A, G, V, L, and I,
X³ is selected from A, G, V, L, I, Q, N, D, E, S, T, M, H, K, and R; and/or
X⁴ to X⁶ are independently selected from A, G, V, L, I, Q, N, D, E, S, T, and M.

### EMBODIMENT 9

The effector polypeptide of any one of embodiments 1 to 8, wherein said effector polypeptide comprises
the amino acid sequence ESNLGWLCLLLLPIPLIVWVKRK (SEQ ID NO:3),
the amino acid sequence QSNLGWLCLLLLPIPLIVWVKRK (SEQ ID NO:4), the amino acid sequence ASNLGWLCLLLLPIPLIVWVKRK (SEQ ID NO:5), the amino acid sequence RSNLGWLCLLLLPIPLIVWVKEE (SEQ ID NO:33), or the amino acid sequence ESNLGWLCLLLLPIPLIVWVKEE (SEQ ID NO:34).

### EMBODIMENT 10

The effector polypeptide of any one of embodiments 1 to 9, wherein said effector polypeptide further comprises at least one of the amino acid sequences of SEQ ID NOs: 6 to 10 as N-terminal sequence(s); and/or at least one of the amino acid sequences of SEQ ID NOs: 11 to 13 as C-terminal sequence(s).

### EMBODIMENT 11

The effector polypeptide of any one of embodiments 1 to 10, wherein said effector polypeptide comprises, preferably consists of, in the order of N-terminus to C-terminus, the amino acid sequences of
(I) SEQ ID NO:1-SEQ ID NO:11;
(II) SEQ ID NO:1-SEQ ID NO:11-SEQ ID NO:12;
(III) SEQ ID NO:1-SEQ ID NO:11-SEQ ID NO:12-SEQ ID NO:13;
(IV) SEQ ID NO:6-SEQ ID NO:1-SEQ ID NO:11-SEQ ID NO:12;
(V) SEQ ID NO:7-SEQ ID NO:6-SEQ ID NO:1-SEQ ID NO:11-SEQ ID NO:12;
(VI) SEQ ID NO:8-SEQ ID NO:7-SEQ ID NO:6-SEQ ID NO:1-SEQ ID NO:11-SEQ ID NO: 12;
(VII) SEQ ID NO:9-SEQ ID NO:8-SEQ ID NO:7-SEQ ID NO:6-SEQ ID NO:1-SEQ ID NO: 11-SEQ ID NO:12;
(VIII) SEQ ID NO:10-SEQ ID NO:9-SEQ ID NO:8-SEQ ID NO:7-SEQ ID NO:6-SEQ ID NO: 1-SEQ ID NO:11-SEQ ID NO:12;
(IX) SEQ ID NO:33-SEQ ID NO:11;
(X) SEQ ID NO:33-SEQ ID NO:11-SEQ ID NO:12;
(XI) SEQ ID NO:33-SEQ ID NO:11-SEQ ID NO:12-SEQ ID NO:13;
(XII) SEQ ID NO:6-SEQ ID NO:33-SEQ ID NO:11-SEQ ID NO:12;
(XIII) SEQ ID NO:7-SEQ ID NO:6-SEQ ID NO:33-SEQ ID NO:11-SEQ ID NO:12;
(XIV) SEQ ID NO:8-SEQ ID NO:7-SEQ ID NO:6-SEQ ID NO:33-SEQ ID NO:11-SEQ ID NO: 12;
(XV) SEQ ID NO:9-SEQ ID NO:8-SEQ ID NO:7-SEQ ID NO:6-SEQ ID NO:33-SEQ ID NO: 11-SEQ ID NO:12;
(XVI) SEQ ID NO:10-SEQ ID NO:9-SEQ ID NO:8-SEQ ID NO:7-SEQ ID NO:6-SEQ ID NO: 33-SEQ ID NO:11-SEQ ID NO:12;
(XVII) SEQ ID NO:34-SEQ ID NO:11;
(XVIII) SEQ ID NO:34-SEQ ID NO:11-SEQ ID NO:12;
(IX) SEQ ID NO:34-SEQ ID NO:11-SEQ ID NO:12-SEQ ID NO:13;
(XX) SEQ ID NO:6-SEQ ID NO:34-SEQ ID NO:11-SEQ ID NO:12;
(XXI) SEQ ID NO:7-SEQ ID NO:6-SEQ ID NO:34-SEQ ID NO:11-SEQ ID NO:12;
(XXII) SEQ ID NO:8-SEQ ID NO:7-SEQ ID NO:6-SEQ ID NO:34-SEQ ID NO:11-SEQ ID NO: 12;
(XXIII) SEQ ID NO:9-SEQ ID NO:8-SEQ ID NO:7-SEQ ID NO:6-SEQ ID NO:34-SEQ ID NO: 11-SEQ ID NO:12; or
(XXIV) SEQ ID NO:10-SEQ ID NO:9-SEQ ID NO:8-SEQ ID NO:7-SEQ ID NO:6-SEQ ID NO: 34-SEQ ID NO:11-SEQ ID NO:12.

### EMBODIMENT 12

The effector polypeptide of any one of embodiments 1 to 10, wherein said effector polypeptide comprises, preferably consists of, the amino acid of SEQ ID NO:32, SEQ ID NO: 31, SEQ ID NO:30, SEQ ID NO: 14, or SEQ ID NO: 15.

### EMBODIMENT 13

The effector polypeptide of any one of embodiments 1 to 11, wherein said effector polypeptide has a decreased binding affinity to glycosphingolipids, preferably ganglioside GM2 (CAS No. 19600 Jan. 2) and/or ganglioside GM3 (CAS No. 54827-14-4), and/or to phosphoinositides, preferably phosphatidylinositol 4,5-bisphosphate (PI(4,5)P₂, CAS No. 245126-95-8) and/or phosphatidylinositol (3,4,5)-trisphosphate (PI(3,4,5)P₃.

### EMBODIMENT 14

The effector polypeptide of any one of embodiments 1 to 13, wherein said effector polypeptide has the activity of reducing pro-tumorigenic signaling in cancer cells.

### EMBODIMENT 15

The effector polypeptide of any one of embodiments 1 to 14, wherein said effector polypeptide has the activity of inducing apoptosis in a cancer cell or in an activated immune cell.

### EMBODIMENT 16

The effector polypeptide of any one of embodiments 1 to 15, wherein said effector polypeptide is a non-naturally occurring polypeptide.

### EMBODIMENT 17

The effector polypeptide of any one of embodiments 1 to 16, wherein said amino acid is an alpha amino acid, preferably an L-alpha amino acid.

### EMBODIMENT 18

The effector polypeptide of any one of embodiments 1 to 17, wherein said amino acid is a proteinogenic amino acid.

### EMBODIMENT 19

A polynucleotide comprising a nucleic acid sequence encoding an effector polypeptide according to any one of embodiments 1 to 18.

### EMBODIMENT 20

The polynucleotide of embodiment 17, wherein said polynucleotide is comprised in an expression construct.

### EMBODIMENT 21

The polynucleotide of embodiment 19 or 20, wherein said polynucleotide is comprised in a vector.

### EMBODIMENT 21

A host cell comprising the effector polypeptide according to any one of embodiments 1 to 16 and/or the polynucleotide according to any one of embodiments 19 to 21.

### EMBODIMENT 23

The host cell of embodiment 22, wherein said host cell produces viral particles and/or liposomes comprising the effector polypeptide according to any one of embodiments 1 to 18 and/or the polynucleotide according to any one of embodiments 19 to 21.

### EMBODIMENT 24

A pharmaceutical composition comprising (A) an active agent selected from the list consisting of an effector polypeptide according to any one of embodiments 1 to 18, a polynucleotide according to any one of embodiments 19 to 21, and/or a host cell according to embodiment 22 or 23 and (B) an excipient.

### EMBODIMENT 25

The pharmaceutical composition of embodiment 24, wherein said excipient is a viral particle and/or a lipid vesicle.

### EMBODIMENT 26

The pharmaceutical composition of embodiment 24 or 25, wherein said pharmaceutical composition comprises a polynucleotide according to any one of embodiments 19 to 21 comprised in a viral particle.

### EMBODIMENT 27

The pharmaceutical composition of embodiment 25 or 26, wherein said viral particle is an adeno associated virus particle, a retrovirus particle, or an adenovirus particle.

### EMBODIMENT 28

An effector polypeptide according to any one of embodiments 1 to 18, a polynucleotide according to any one of embodiments 19 to 21, a host cell according to embodiment 22 or 23, and/or a pharmaceutical composition according to any one of embodiments 24 to 27, for use in medicine.

### EMBODIMENT 29

An effector polypeptide according to any one of embodiments 1 to 18, a polynucleotide according to any one of embodiments 19 to 21, a host cell according to embodiment 22 or 23, and/or a pharmaceutical composition according to any one of embodiments 24 to 27, for use in treating and/or preventing cancer, inflammatory disease, or acute or chronic neurodegenerative disease.

### EMBODIMENT 30

The effector polypeptide for use of embodiment 29, wherein said cancer is a CD 95-expressing cancer and/or showing a high degree of immune infiltration, preferably is brain cancer, more preferably glioblastoma, or pancreatic cancer, preferably pancreatic ductal adenocarcinoma (PDAC).

### EMBODIMENT 31

A method of treating and/or preventing cancer, inflammatory disease, or acute or chronic neurodegenerative disease in a subject in need of such treatment comprising
(a) contacting said subject with an effector polypeptide according to any one of embodiments 1 to 18, a polynucleotide according to any one of embodiments 19 to 21, a host cell according to embodiment 22 or 23, and/or a pharmaceutical composition according to any one of embodiments 24 to 27; and
(b) thereby treating and/or preventing cancer, inflammatory disease, or acute or chronic neurodegenerative disease.

### EMBODIMENT 32

The subject matter of any one of embodiments 29 to 31, wherein said treating and/or preventing further comprises administration of at least one inhibitor of a receptor tyrosine kinase.

### EMBODIMENT 33

The subject matter of embodiment 32, wherein said wherein said receptor tyrosine kinase is epidermal growth factor receptor (EGFR).

### EMBODIMENT 34

The subject matter of embodiment 32 or 33, wherein said inhibitor of a receptor tyrosine kinase comprises, preferably is, Erlotinib, Afatinib, Dacomitinib, Gefitinib, Lapatinib, Neratinib, Osimertinib, and/or Vandetanib.

### EMBODIMENT 35

A kit comprising an effector polypeptide according to any one of embodiments 1 to 18, a polynucleotide according to any one of embodiments 19 to 21, a host cell according to embodiment 22 or 23, and/or a pharmaceutical composition according to any one of embodiments 24 to 27 in a housing.

### EMBODIMENT 36

The kit of embodiment 35, wherein said kit further comprises at least of excipient and/or a means of administration.

### EMBODIMENT 37

Use of an effector polypeptide according to any one of embodiments 1 to 18, a polynucleotide according to any one of embodiments 19 to 21, a host cell according to embodiment 22 or 23, and/or a pharmaceutical composition according to any one of embodiments 24 to 27 for inducing apoptosis in a host cell. Embodiment 38: The use of embodiment 37, wherein said use is an in vitro use.

### EMBODIMENT 39

A method for identifying an inhibitor of an interaction of CD95 with at least one membrane lipid (interaction inhibitor), comprising
(a) contacting a host cell comprising CD95 or a fragment thereof and at least one membrane lipid with a candidate inhibitor;
(b) determining interaction between CD95 or said fragment thereof and membrane lipids;
(c) comparing the interaction determined in step (b) to a control interaction; and,
(d) based on the comparison of step (d), identifying an interaction inhibitor.

### EMBODIMENT 40

The method of embodiment 39, wherein said CD95 or fragment thereof comprises the transmembrane domain of CD95 or a variant thereof, preferably comprises SEQ ID NO: 1.

### EMBODIMENT 41

The method of embodiment 39 or 40, wherein said control interaction is determined in a host cell not contacted with the candidate inhibitor and/or wherein said control interaction is determined in a host cell comprising the effector polypeptide according to any one of embodiments 1 to 18.

### EMBODIMENT 42

The method of any one of embodiments 39 to 41, wherein said CD95 or fragment thereof is labeled with a first detector compound, preferably a first fluorescent label; and/or wherein said at least one membrane lipid is labeled with a second detector compound, preferably a second fluorescent label.

### EMBODIMENT 43

The method of any one of embodiments 39 to 42, wherein said at least one membrane lipid comprises, preferably is, a ganglioside, preferably ganglioside GM2 and/or ganglioside GM3.

### EMBODIMENT 44

An inhibitor of interaction of CD95 with at least one membrane lipid for use in medicine, preferably for use in treating cancer, inflammatory disease, or acute or chronic neurodegenerative disease.

### EMBODIMENT 45

The inhibitor for use of embodiment 44, wherein said inhibitor is a direct inhibitor of interaction of CD95 with at least one membrane lipid, preferably is an effector polypeptide according to any one of embodiments 1 to 18.

### EMBODIMENT 46

The inhibitor for use of embodiment 44 or 45, wherein said inhibitor is an indirect inhibitor of interaction of CD95 with at least one membrane lipid, preferably an inhibitor of ganglioside biosynthesis, preferably of ganglioside GM2 and/or ganglioside GM3 biosynthesis.

### EMBODIMENT 47

A method of identifying a subject suffering from cancer, inflammatory disease, or acute or chronic neurodegenerative disease to be susceptible to treatment with an inhibitor of interaction of CD95 with at least one membrane lipid, said method comprising
(A) determining CD95 expression and/or dependency from receptor tyrosine kinase activity of disease-mediating cells of said subject, and
(B) identifying a subject susceptible to treatment with an inhibitor of interaction of CD95 with at least one membrane lipid based on the determination of step (A).

### EMBODIMENT 48

The method of embodiment 47, wherein a subject susceptible to treatment with an inhibitor of interaction of CD95 with at least one membrane lipid is identified if said disease-mediating cells are found to express CD95 and/or to be dependent on receptor tyrosine kinase activity in step (A).

### EMBODIMENT 49

A method of treating a subject suffering from cancer, inflammatory disease, or acute or chronic neurodegenerative disease with an inhibitor of interaction of CD95 with at least one membrane lipid, said method comprising
steps (A) and (B) of the method of embodiment 45 or 48, and further step (C) treating a subject identified to be susceptible to treatment by an inhibitor of interaction of CD95 with at least one membrane lipid by administering an inhibitor of interaction of CD95 with at least one membrane lipid.

All references cited in this specification are herewith incorporated by reference with respect to their entire disclosure content and the disclosure content specifically mentioned in this specification.

### FIGURE LEGENDS

FIG. 1: A) Contact occupancy of the CD95 transmembrane domain for specified lipid species. The numbers correspond to amino acid positions in the TMD of CD95 sequence used for atomistic simulations; Chol: Cholesterol, PSM: palmitoylsphingomyelin, POPI: 1-palmitoyl-2-oleoyl-sn-glycero-3-phosphoinositol, PI(4,5)PI₂: phosphatidylinositol 4,5-bisphosphate, PI(3,5)PI₂: phosphatidylinositol 3,5-bisphosphate, PI(3,4,5)PI₃: phosphatidylinositol 3,4,5-trisphosphate, GM1: ganglioside, GM2: ganglioside GM2, GM3: ganglioside GM3, GD3:ganglioside GD3, Gb3: globotriaosylceramide, GT1a: ganglioside GT1a, GT1b: ganglioside GT1b; Interactions labeled with a cross (x) are in a frequency range of from 0 to 0.5 (low frequency), others are in the range of from <0.5 to 1 (high frequency); white indicates no applicable analysis due to asymmetric lipid assembly of plasma membrane. In accordance, the cell exterior would be in FIG. 1 left of R171, while the cytoplasm would be right of T198; B), C) Contact occupancy of the CD95 transmembrane domain or muteins thereof for specified lipid species; B) gangliosides (GM2 and GM3), C) phosphatidylinositols (PI(4,5)PI₂) and PI(3,4,5)PI₃).

FIG. 2: CD95KO cells show decreased basal pAkt levels and hyperactivation of EGFR upon EGF stimulation. Immunoblot analyses of extracts from cells indicated at the top of the Figure with antibodies indicated on the right.

FIG. 3: Apoptosis analysis of transmembrane domain (TMD) wt and TMD mutant CD95 overexpressing T98G cells. The TMD mutant CD95 comprises an R171Q exchange. CD95L: stimulation by growth on a mCD95L coated membrane, DOPC: growth on control membrane (no CD95L); wt: CD95 wt overexpression; mut: CD95 R171Q overexpression.

FIG. 4: Modeling of lipid interaction of a triple R171E/R192E/K193E mutant CD95 transmembrane region. Atomistic (AA) molecular dynamics simulations (MDS) of CD95 in its wild type (WT) and triple mutant form (3M; R171E, R192E, and K193E). The protein fragment used for the simulations spans from residues E168 to T198, embedded in an asymmetric bilayer containing 128 phospholipid molecules (64 lipids per leaflet). The upper leaflet contains 10 mol % GM3 (10 molecules), whereas the lower leaflet contains 10 mol % PI(4,5)P₂ molecules. The systems were simulated for 2 microseconds in triplicate repetition, but the first microsecond has been considered equilibration time and then discarded from the analysis. A)-C) contact occupancy between POPC (A)), GM3 (B)), PI(4,5)P₂ (C)) against every amino acid of the protein, as the wild-type (upper lines) and as the 3M (lower lines); D) shows, instead, the average number of contacts between each lipid type and the full protein during the simulated time.

FIG. 5: Changes of lipid composition of membranes of cells in the presence of CD95 variants. A) Shown are average mol % concentrations of the indicated lipid classes; Abbreviations: WT: wildtype CD95; KO: CD95-knockout; sM: single mutant (R171E), dM: double mutant (R192E/K193E); tM: triple mutant (R171E/R192E/K193E); PC: phosphatidylcholines; PE: phosphatidylethanolamines, PS: phosphatidylserines, PI: phosphatidylinositoles, SM: sphingomyelins, CE: cholesteryl esters. “a”X relates to acyl-X, e.g. aPC is Acyl-phosphatidylcholines; “e”X relates to Ether-X or X containing one odd-chain fatty acyl species. B) Clustering of cells based on lipid profile changes, UMAP representation. C) average cell size as cell area of cells expressing variants of CD95.

FIG. 6: Effect of CD95 variants on cell cycle. Shown is the percentage of cells in G2, S, or G1 phase, respectively, in cell overexpressing effector polypeptides. Double and triple mutation variants induced a strong G1 arrest of cells. Abbreviations as in FIG. 5.

FIG. 7: Effect of CD95 variants on cell signaling; shown is relative content in pAkt and pEGFR in cells transfected to overexpress effector polypeptides as indicated. Abbreviations as in FIG. 5.

FIG. 8: Effect of CD95 variants on endocytotic activity of cells. Shown is the relative transferrin uptake of treated cells over time, i.e. after 10, 20, and 30 min. Abbreviations as in FIG. 5.

FIG. 9: Impact of CD95 mutants on cell death. Shown are fractions of living cells (A) and dead (B) T36 cells after expression of the indicated constructs.

FIG. 10: Impact of CD95 mutants on activation of Akt and EGFR. Protein samples were analysed by western blot for pAKT (ser 473), pEGFR and pERK and their corresponding total (pan) antibodies: (A) shows fractions of phosphorylated Akt (pAkt) in total Akt, and (B) shows fractions of phosphorylated EGFR (pEGFR) in total EGFR. Abbreviations as in FIG. 5.

FIG. 11: Impact of CD95 mutants on apoptosis (caspase-8 cleavage). Protein lysates were analysed for the level of cleaved and total caspase-8. Total caspase was observed at 60 kDa followed by cleaved caspase pro-domain p44/p43 (containing the p18) and also the p18 subunit separately. Bar show, in each case from left to right, relative expression of total caspase, p44, p43, and p18.

The following Examples shall merely illustrate the invention. They shall not be construed, whatsoever, to limit the scope of the invention.

### EXAMPLE 1

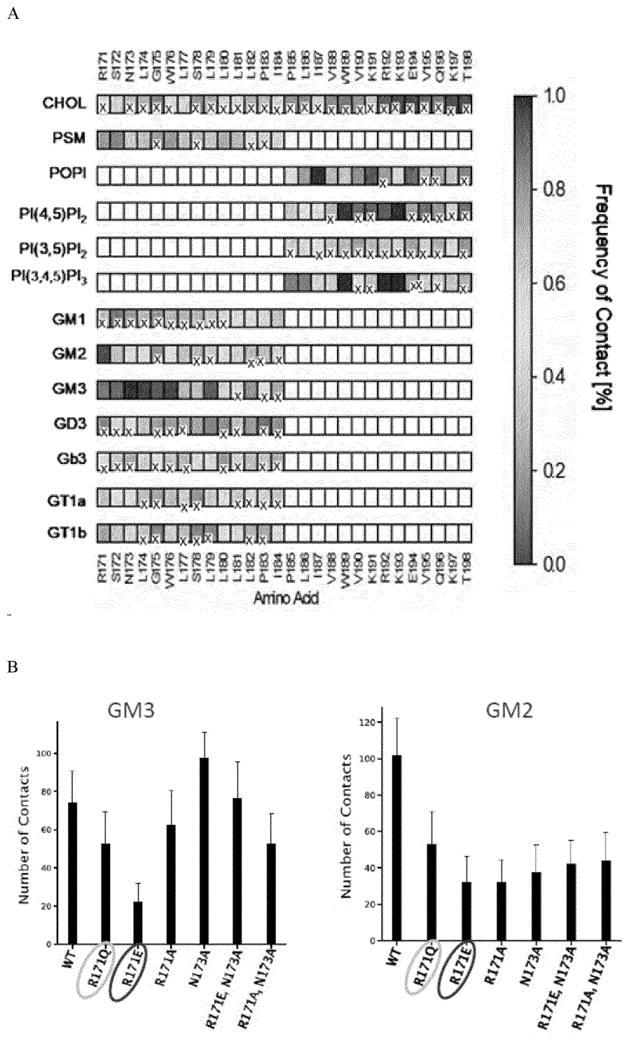

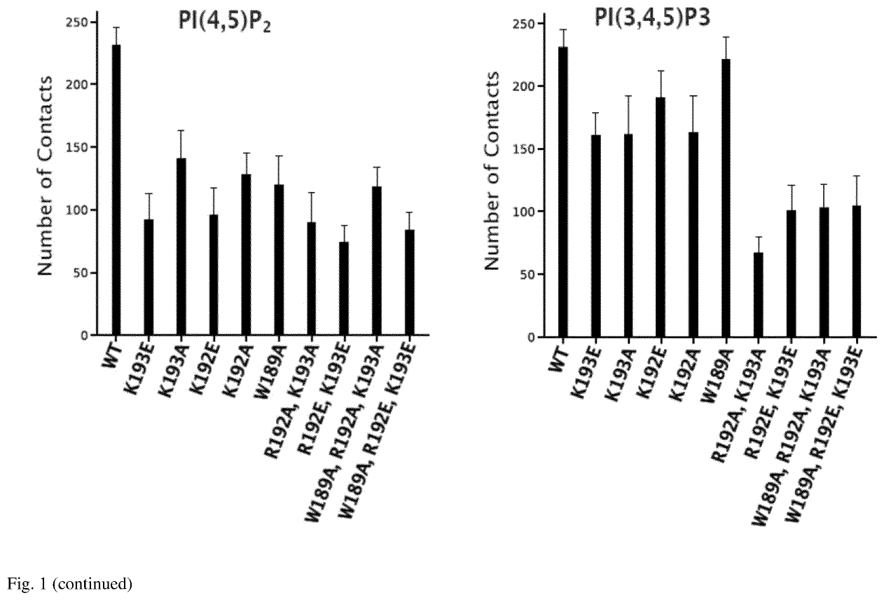

### Contact Occupancy for Specified Lipid Species (FIG. 1)

The contact (defined as a distance <0.6 nm) analysis has been performed over the last 800 nanosecond of each repeat. For these atomistic molecular dynamics (MD) simulations, we used the truncated structure of CD95 (PDB ID: 2NA7) from residue 171 to 198 in its monomeric form (Fu et al. (2016), Mol Cell 61:602-613). Three additional residues (168-170) have been added using the MODELLER software (Fiser and Šali (2003), Methods in Enzymology 374:461-491). The preparation of the CD95 structure for MD simulations involved a submission of the protein data bank (PDB) structure to CHARMM-GUI web site, where we modelled missing the N-and C-terminal groups as neutral residues (Lee et al. (2016), J. Chem. Theory Comput. 12 (1): 405-413).

CD95 was inserted in two different POPC-based lipid bilayer containing 10 mol % of GM2 and GM3 glycosphingolipids, respectively. The systems were first hydrated and neutralized by an appropriate number of counterions, followed by the addition of 150 mM of potassium chloride. to mimic the experimental conditions. For protein, lipid and ions we used the CHARMM36m force field, whereas water was described using the standard CHARMM36 TIP3P model (Huang et al. (2017), Nat Methods 14 (1): 71-73).

All systems were energy minimized using the steepest descent algorithm. After the minimization, an equilibration under NpT conditions was performed. In this step, protein was restrained in all dimensions, whereas lipid molecules were restrained only in the z-axis. The Nose-Hoover thermostat was used to maintain the temperature at 310 K with a time constant of 1.0 ps (Evans and Holian (1985), J. Chem. Phys. 83:4069). The pressure of 1 atm was kept constant using the Berendsen barostat with a time constant set to 5.0 ps and isothermal compressibility to a value of 4.5×10⁻⁵ bar⁻¹ (Berendsen et al. (1984), J. Chem. Phys. 81:3684). The semi-isotropic pressure coupling scheme was used. For neighbor searching, we used the Verlet scheme with a cut-off distance of 20 steps. The electrostatic interactions were calculated using the PME method (Darden et al. (1993), J. Chem. Phys. 98:10089). The cut-off length of 1.2 nm was used for both electrostatic and van der Waals interactions. Periodic boundary conditions were applied in all directions. For the production MD runs, we removed all the restraints and used the Parrinello-Rahman barostat (Nosé and Klein (1983), Molecular Physics, 50 (5): 1055-1076, Taylor & Francis). Other input parameters for production MD simulations were the same as those used under NpT equilibration. The simulations were carried out using an integration time step of 2 fs until they reached 1 μs.

All simulations were performed using the GROMACS 2020.2 simulation package (Abraham et al. (2015), SoftwareX 1-2:19-25). All analyses were done for the last 800 ns of the simulation trajectories (unless stated otherwise) using standard GROMACS tools and in-house scripts.

The atomistic simulations predicted lipid interaction sites on the transmembrane domain (TMD) of CD95 and the corresponding lipid partners are shown in the heat map of FIG. 1. N-terminal residue R171 and the C-terminal residues R192/K193 are predicted to be critical for the selectivity of lipid-protein interactions for CD95. As to the lipid type, gangliosides GM2 and GM3, and sphingomyelin bind to CD95 in the outer leaflet. PI(4,5)P₂ and PI(3,4,5)P₃ bind to CD95 in the inner leaflet of the membrane.

### EXAMPLE 2

### Lipid Profile Analysis

We generated CD95KO cells via CRISPR/Cas9. CD95KO and their parental cells were used to extract the total lipids, which were further subjected to quantitative mass spectrometric lipid analysis. Lipidomics analysis revealed significant changes in certain lipid species, including phosphatidylcholine (PC), cholesterol (Chol) and sphingolipid species like sphingomyelin (SM) and dihexosylceramides (Hex2Cer). Specifically, the levels of SM increased while Hex2Cer decreased when CD95 was knocked out. These observations were also confirmed in other GBM patient-derived cells. Thus, CD95 elimination by CRISPR/Cas9 targeting identifies a hitherto unknown regulatory impact of CD95 on the global lipid composition.

### EXAMPLE 3

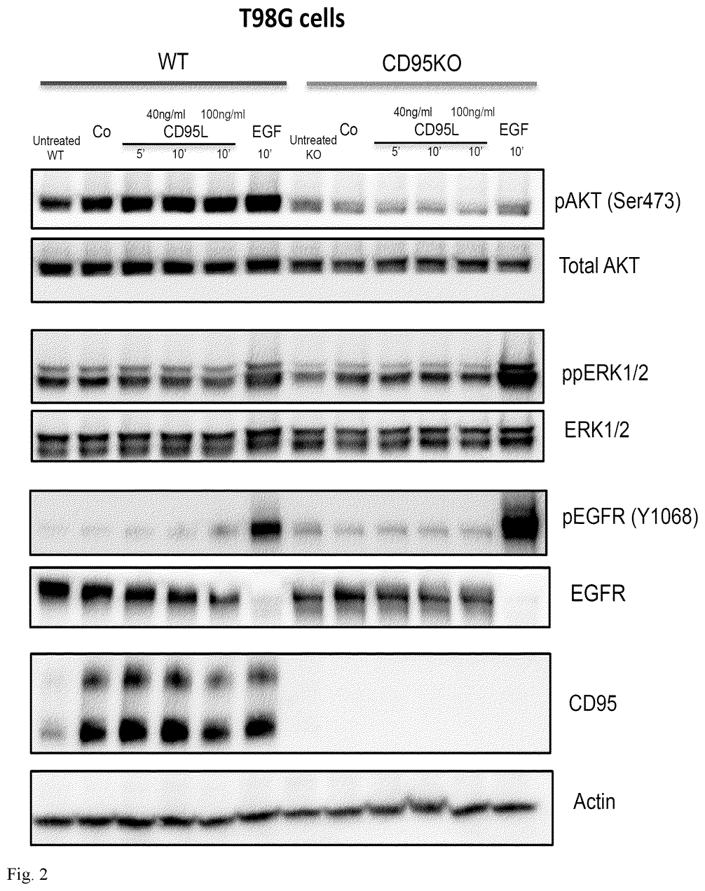

### Signaling Pathways in CD95-KO cells (FIG. 2)

T98G CD95KO cell were generated as described in Example 2. In T98G CD95KO cells, pAKT basal levels were lower compared to wt cells. Yet, EGF elicited higher pEGFR and ppERK1/2levels in CD95KO than in wt cells. Total AKT, ERK1/2 and EGFR signals were comparable between wt and CD95KO cells. Cells were treated with interferon-gamma (IFNγ) for 17 hours to enhance the expression of CD95. Untreated cells were used as control. Actin was used as a loading control. Stimulation time (in minutes) and concentration of ligands are indicated in the figure.

Notably, CD95KO cells exhibited lower phosphorylation of AKT. This seemed to be compensated by enhanced EGFR phosphorylation in response to EGF.

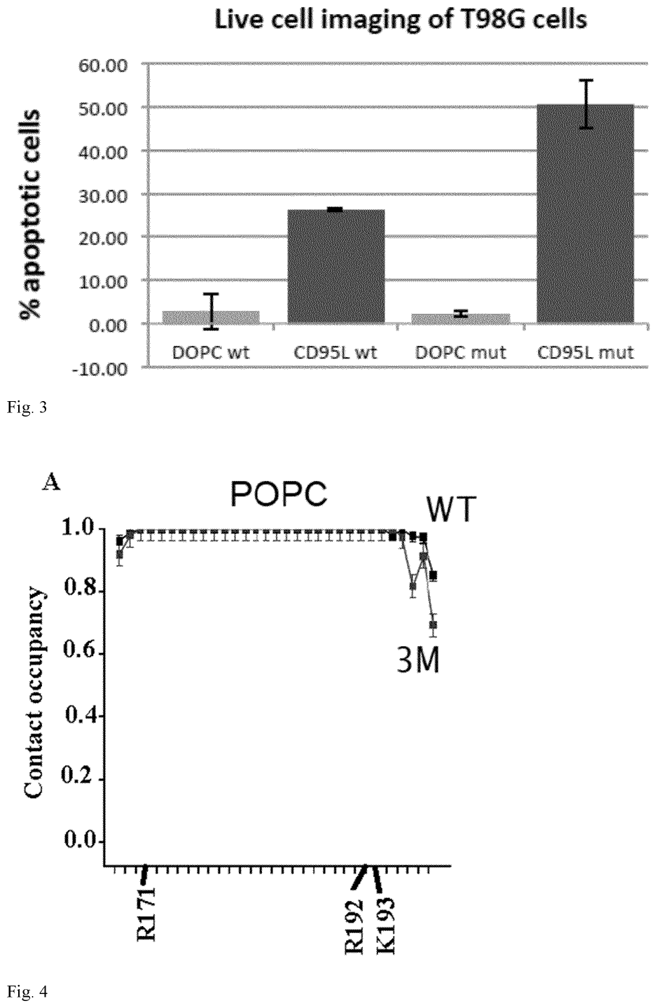

### EXAMPLE 4: (FIG. 3)

In glycomics data, GM2 was found to be abolished in CD95KO cells, corresponding to the preferential binding lipid partner to R171 residue from atomistic simulations. This site was mutated to glutamine (R171Q, “mut” in FIG. 3). The cells were transfected to either overexpress wt or mutant CD95, and were analyzed on a CD95L supported membrane for apoptosis induction.

As shown in FIG. 3, exchanging of the amino acid interacting with GM2 (FIG. 1A)) for a less interacting amino acid (FIG. 1B)) caused a massive increase of apoptosis after CD95L stimulation. Notably, the cells were wildtype-CD95 expressing cells overexpressing the respective CD95 variant, i.e., apoptosis induction was increased with the R171Q mutein despite the presence of wt CD95 in the cells.

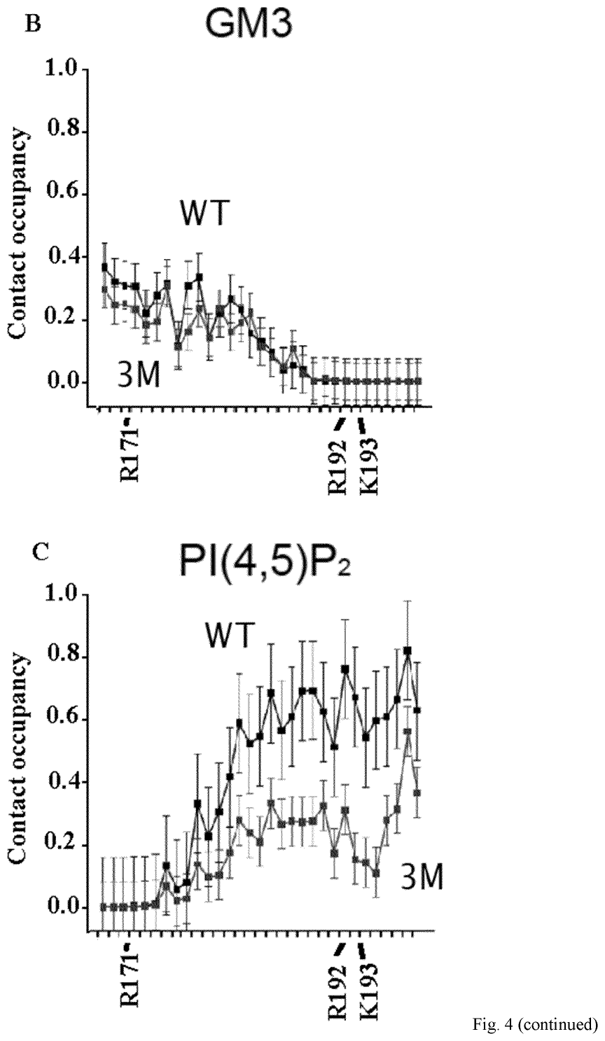

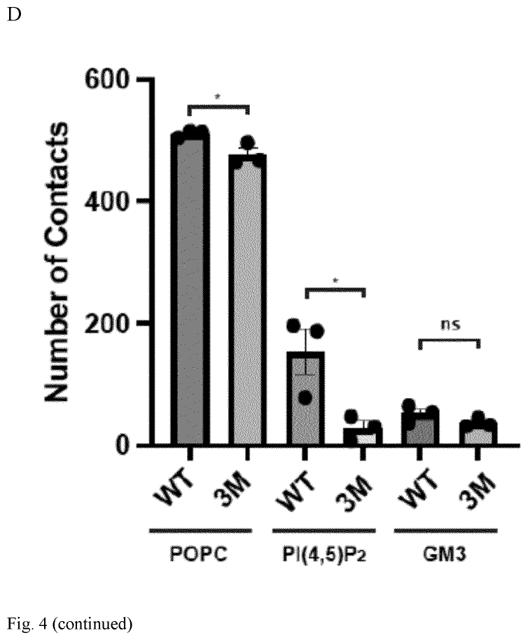

### EXAMPLE 5 (FIG. 4)

### Modeling of Lipid Interaction of Triple Mutated TM Region (3M)

FIG. 4C) clearly shows a drastic reduction of PI(4,5)P₂-CD95 interaction in the 3M variant compared to the wildtype, especially at the level where two of the mutations are located (R192E, K193E). In the case of GM3-CD95 (FIG. 4B)), the difference between WT and 3M are not significant. FIG. 4 D) shows the total number of contacts and essentially confirms results of Fag. 4 A) to C). Notably, when PI(4,5)P₂ molecules interact with CD95, the long acyl chains (18:0/20:4) surround the protein pushing the other protein's end into the membrane hydrophobic core. This forces R171 to be less contact surrounding water and to more contact with the negative P atoms of POPC, which makes R171 less accessible for GM3 head groups.

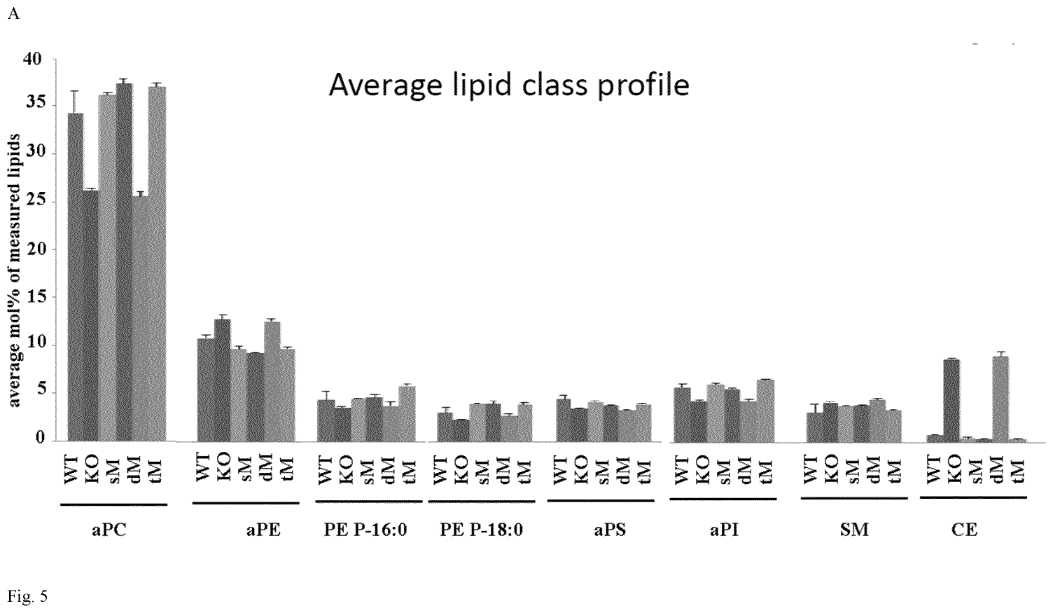

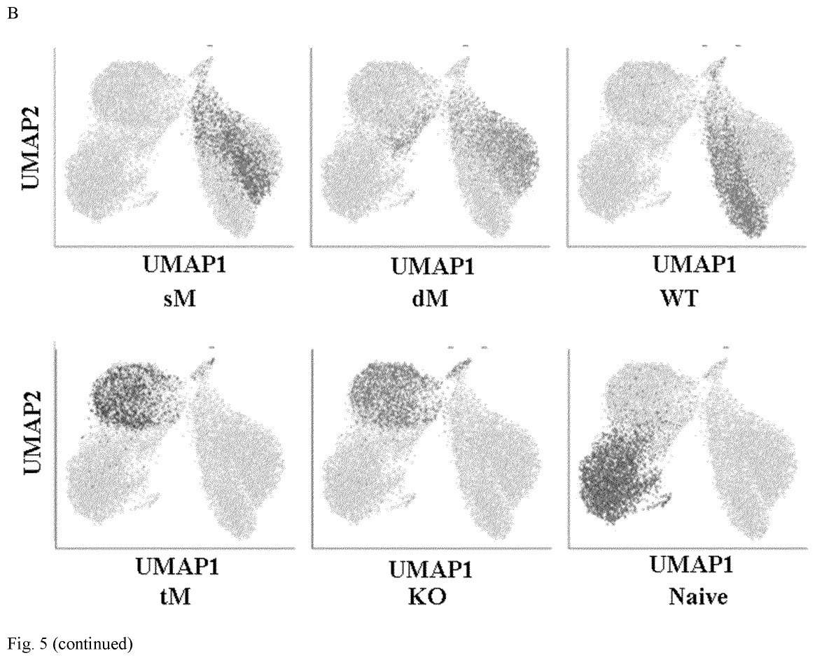

### EXAMPLE 6 (FIG. 5)

### Effects of CD95 Mutants on Membrane Composition of Cells

Indicated variants were overexpressed using lentiviral vectors two weeks prior to lipidomics profiling. In particular cells expressing the double mutant or the triple mutant, as well as CD95 mutant cells, showed distinct changes in membrane lipid composition (FIG. 5A). Notably, the single and the double mutant clustered similarly, while, surprisingly, the triple mutant showed very similar clustering to the KO cells. Also, average cell size was decreased in cells expressing the triple mutant CD95, very similar to CD95 KO cells.

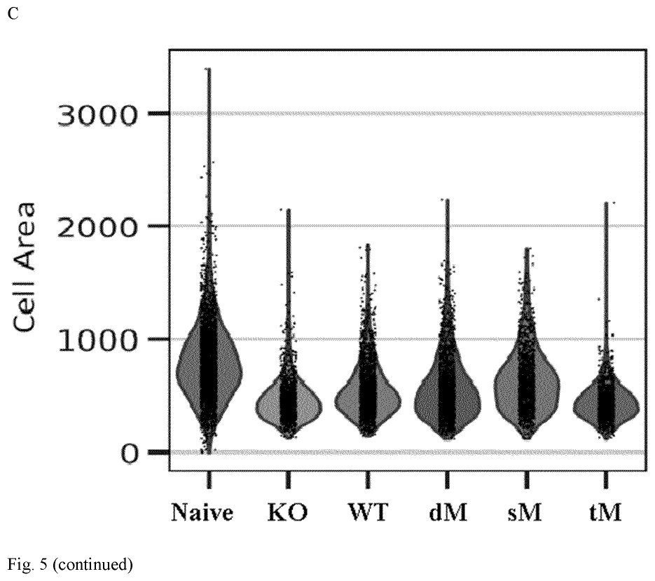

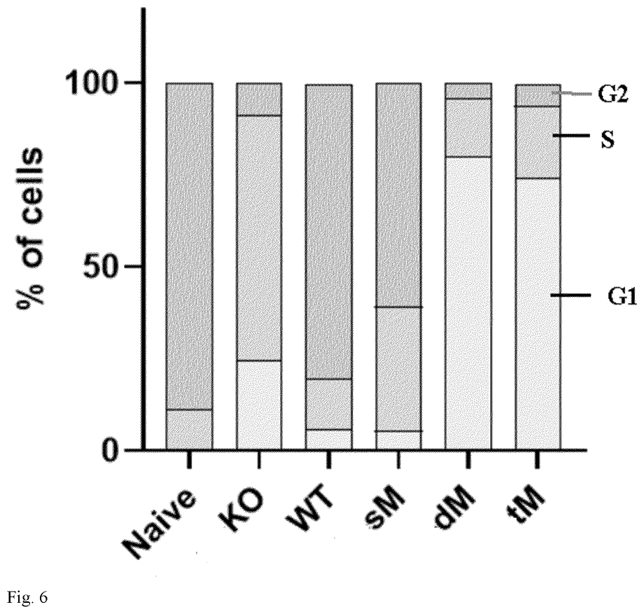

### EXAMPLE 7 (FIG. 6)

### Cell Cycle Effects

Indicated variants were overexpressed using lentiviral vectors two weeks prior to cell cycle assessment. As shown in FIG. 6, the double and triple mutants caused a G1 arrest in cells.

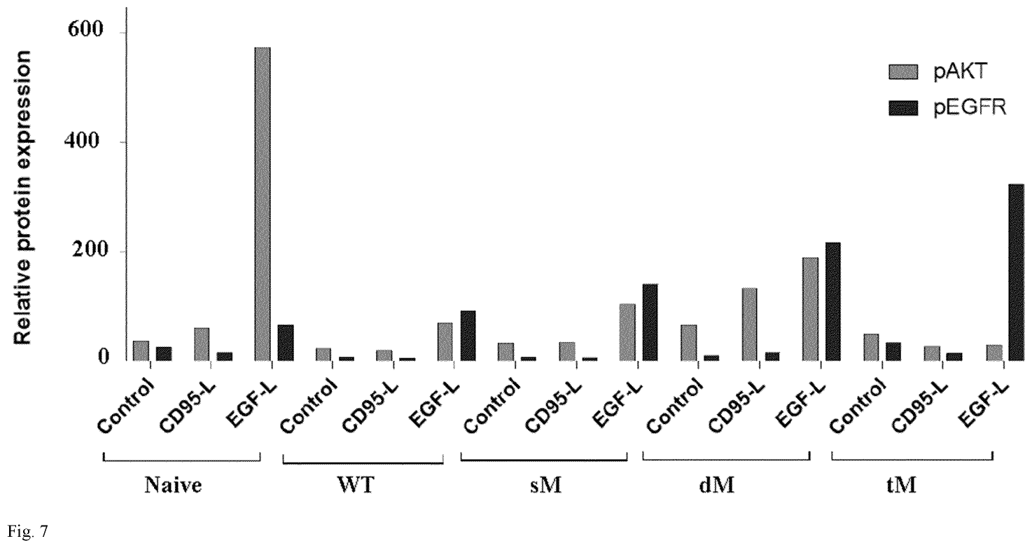

### EXAMPLE 8 (FIG. 7)

### Cell Signaling Effects

Indicated variants were overexpressed using lentiviral vectors two weeks prior to treatment. Cells were left untreated (co) or exposed to 20 ng/ml of CD95-ligand or 100 ng/ml of EGF-L for 10 minutes and the fraction phosphorylation among total Akt and EGFR measured by Western Blot. As shown in FIG. 7, cells overexpressing the triple mutant show reduced Akt phosphorylation (Ser 473), and increased EGFR phosphorylation.

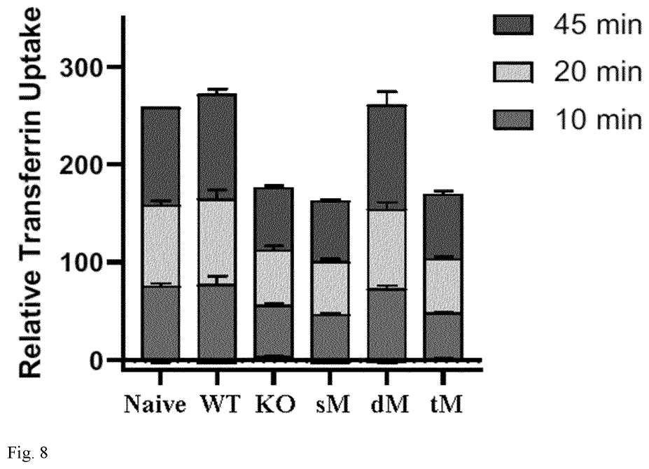

### EXAMPLE 9 (FIG. 8)

### Endocytosis

Indicated variants were overexpressed using lentiviral vectors two weeks prior to assessment of the endocytotic activity by a transferrin uptake assay. Transferrin uptake was assessed after the indicated time points via flow cytometry. CD95 KO cells, but also single or triple mutant overexpressing cells showed significantly reduced transferrin uptake.

### EXAMPLE 10

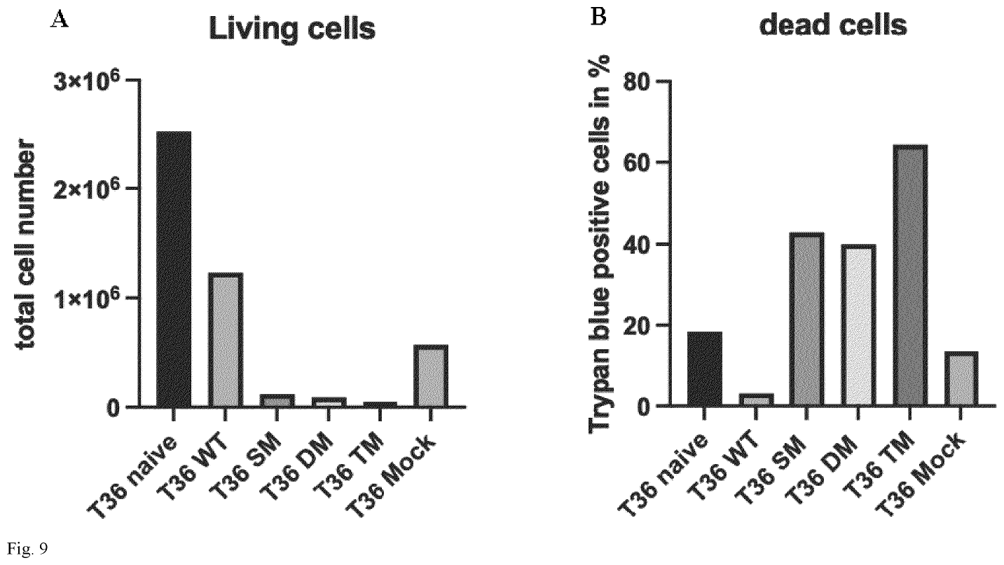

### Impact of CD95 Mutants on Cell Death (FIG. 9)

T36 patient-derived glioblastoma cells were plated out in a 6-well plate. The next day, cells were transduced with the following AAVs (10.000 Vg/cell): CD95-WT (WT), CD95-single mutant (SM), CD95-double mutant (DM), CD95-tripple mutant (TM) and YFP (mock, as internal control for AAV transduction). After 72 hours post-transduction, the survival/death rate of the transduced cells was determined by trypan blue uptake. All mutants caused the fraction of dead cells to increase to at least approx. 40%.

### EXAMPLE 11

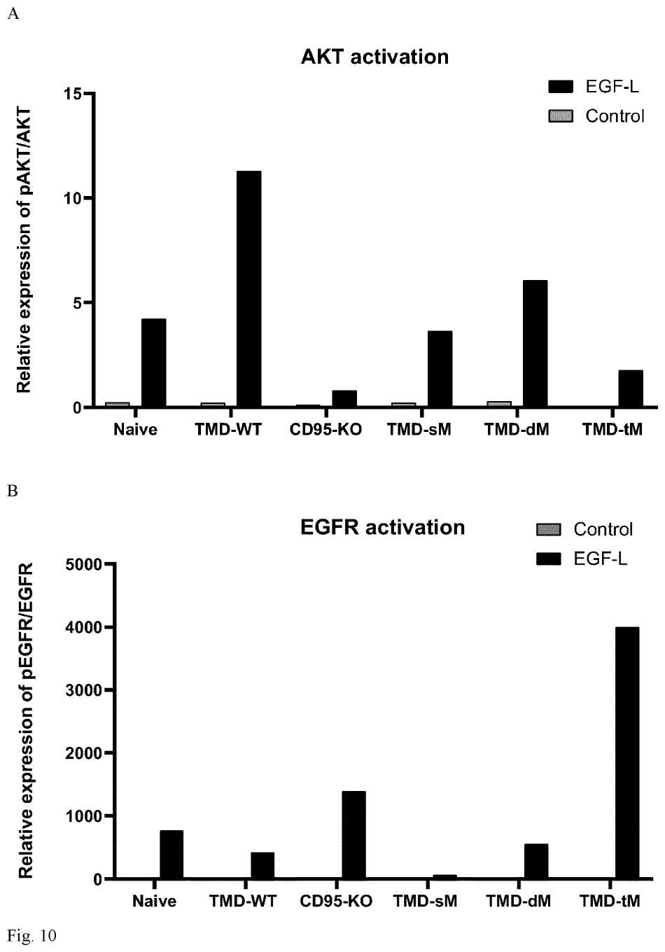

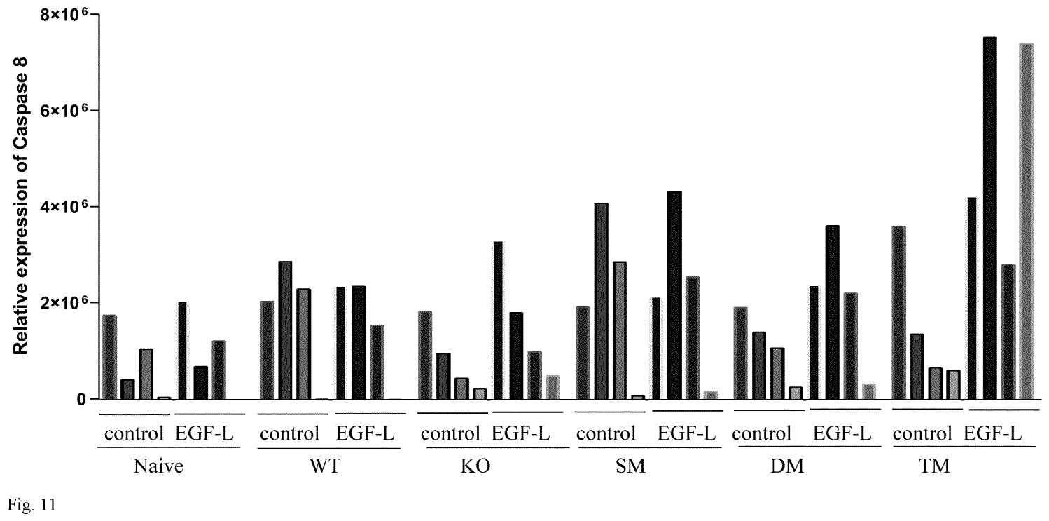

### Impact of CD95 Mutants on Activation of Akt, EGFR and Apoptosis (FIGS. 10 and 11)

T98G cells were serum starved, control (Co) cells were left untreated otherwise stimulated with 100 ng/ml of EGF-L for 10 mins. Protein samples were analysed by western blot for pAKT (ser 473), pEGFR and pERK and the total amount of protein (pan antibody). Relative expression of the protein was calculated where phospho-proteins were normalized to their total proteins. It was found that CD95-KO and the triple mutant (TM) show a decrease in the level of AKT activation, but an increase in the EGFR activation (FIG. 10). TMD is CD95 with the indicated mutation(s). Protein lysates were also analysed for the level of cleaved and total caspase (FIG. 11). A noticeable Caspase 8 cleavage was observed in particular in TM upon EGFR stimulation.

### Non-Standard Literature:

Abraham et al. (2015), SoftwareX 1-2:19-25
Berendsen et al. (1984), J. Chem. Phys. 81:3684
Bethani et al. (2010), EMBO J. 29:2677
Balta et al. (2019), Cell Reports 29 (8): 2295
Balta, Gülcüler (2019), Ph.D. thesis submitted to the University Heidelberg
Darden et al. (1993), J. Chem. Phys. 98:10089
Evans and Holian (1985), J. Chem. Phys. 83:4069
Fiser and Šali (2003), Methods in Enzymology 374:461-491
Fu et al. (2016), Mol Cell 61:602-613
Haberkant & Holthuis (2014), BBA-Mol Cell Biol Lipids 1841 (8): 1022
Hean & Weinberg (2008), RNA and the Regulation of Gene Expression: A Hidden Layer of Complexity, Chapter 1. Caister Academic Press
Huang et al. (2017), Nat Methods 14 (1): 71-73
Kleber et al. (2008), Cancer Cell 13:235-248
Lee et al. (2016), J. Chem. Theory Comput. 12 (1): 405-413
Martin-Villalba et al. (2013), Trends Mol Med 19 (6): 329
Nakano et al. (2021), BBA Biomembranes 1863:186323
Nosé and Klein (1983), Molecular Physics, 50 (5): 1055-1076, Taylor & Francis
Sancho-Martinez & Martin-Villalba (2009); Cell Cycle. 8:838
Svoronos et al. (2020), Mol Pharmaceutics 17:461
WO 01/39804
Yu et al. (2011), J Oleo Sci 60 (10): 537

1. An effector polypeptide comprising
(i) an amino acid sequence at least 70% identical to the amino acid sequence RSNLGWLCLLLLPIPLIVWVKRK (SEQ ID NO:1); and
(ii) an exchange of an amino acid to a non-identical amino acid at at least one position selected from the list consisting of positions 1, 2, 3, 19, 22, and 23 of the amino acid sequence of (i).

2. The effector polypeptide of claim 1, wherein said amino acid exchange(s) is/are selected from the list consisting of R1E, R1Q, R1A, S2A, N3A, R1E/N3A, R1A/N3A, W19A, R22A, R22E, K23A, K23E, R22A/K23A, R22E/K23E, W19A/R22A/K23A, W19A/R22E/K23E, and any combination thereof.

3. The effector polypeptide of claim 1, wherein said amino acid exchange is R1E, R1Q, or R1A.

4. The effector polypeptide of claim 1 comprising at least two or at least three amino acid exchanges.

5. The effector polypeptide of claim 1, wherein said amino acid exchange(s) is/are selected from the list consisting of R1E, R22E, and K23E.

6. The effector polypeptide of claim 1, wherein said effector polypeptide comprises the amino acid sequence X¹X²X³LGWLCLLLLPIPLIVX⁴VKX⁵X⁶ (SEQ ID NO:2), wherein
X¹ is selected from E, Q, A, N, G, V, L, I, S, T, D, and M, preferably is E or Q;
X² is selected from A, G, V, L, and I,
X³ is selected from A, G, V, L, I, Q, N, D, E, S, T, M, H, K, and R; and/or
X⁴ to X⁶ are independently selected from E, A, G, V, L, I, Q, N, D, S, T, and M.

7. The effector polypeptide of claim 1, wherein said amino acid exchanges comprise R1E, R22E, and K23E.

8. The effector polypeptide of claim 1, wherein said effector polypeptide comprises the amino acid sequence RSNLGWLCLLLLPIPLIVWVKEE (SEQ ID NO:33).

9. The effector polypeptide of claim 1, wherein said effector polypeptide comprises the amino acid sequence ESNLGWLCLLLLPIPLIVWVKEE (SEQ ID NO:34).

10. The effector polypeptide of claim 1, wherein said effector polypeptide comprises:
the amino acid sequence ESNLGWLCLLLLPIPLIVWVKRK (SEQ ID NO:3),
the amino acid sequence QSNLGWLCLLLLPIPLIVWVKRK (SEQ ID NO:4), or the
amino acid sequence ASNLGWLCLLLLPIPLIVWVKRK (SEQ ID NO:5).

11. The effector polypeptide of claim 1, wherein said effector polypeptide further comprises:
at least one of the amino acid sequences of SEQ ID NOs: 6 to 10 as N-terminal sequence(s); and/or
at least one of the amino acid sequences of SEQ ID NOs: 11 to 13 as C-terminal sequence(s).

12. The effector polypeptide of claim 1, wherein said effector polypeptide comprises the amino acid of SEQ ID NO: 32, SEQ ID NO: 31, SEQ ID NO:30, SEQ ID NO: 14, or SEQ ID NO: 15.

13. A polynucleotide comprising a nucleic acid sequence encoding an effector polypeptide according to claim 1.

14. A host cell comprising the effector polypeptide according to claim 1.

15. A pharmaceutical composition comprising (A) an active agent being an effector polypeptide according to claim 1 and (B) an excipient.

16. The pharmaceutical composition of claim 15, wherein said excipient is a viral particle and/or a lipid vesicle.

17-20. (canceled)

21. A method of treating and/or preventing cancer, inflammatory disease, or acute or chronic neurodegenerative disease in a subject in need of such treatment comprising:
(a) contacting said subject with an effector polypeptide according to claim 1; and
(b) thereby treating and/or preventing cancer, inflammatory disease, or acute or chronic neurodegenerative disease.

22. The method of claim 21, wherein said cancer is a CD 95-expressing cancer and/or showing a high degree of immune infiltration, preferably is brain cancer, more preferably glioblastoma, or pancreatic cancer, preferably pancreatic ductal adenocarcinoma (PDAC).

23. The method of claim 21, wherein said treating and/or preventing further comprises administration of at least one inhibitor of a receptor tyrosine kinase.

24. The method of claim 23, wherein said inhibitor of a receptor tyrosine kinase is selected from the group consisting of Erlotinib, Afatinib, Dacomitinib, Gefitinib, Lapatinib, Neratinib, Osimertinib, Vandetanib, and any combination thereof.


SEQUENCE LISTING (34 sequences)

SEQ ID NO: 1 - protein, 23 aa, Homo sapiens
RSNLGWLCLLLLPIPLIVWVKRK

SEQ ID NO: 2 - protein, 23 aa, synthetic construct
  REGION 1..23: note = CD95 TMD generic mutein
  VARIANT 1: note = MISC_FEATURE - X is E, Q, A, N, G, V, L, I, S, T, D, or M
  VARIANT 2: note = MISC_FEATURE - X is A, G, V, L, or I,
  VARIANT 3: note = MISC_FEATURE - X is A, G, V, L, I, Q, N, D, E, S, T, M, H, K, or R
  VARIANT 19: note = MISC_FEATURE - X is A, G, V, L, I, Q, N, D, E, S, T, or M
  VARIANT 22: note = MISC_FEATURE - X is A, G, V, L, I, Q, N, D, E, S, T, or M
  VARIANT 23: note = MISC_FEATURE - X is A, G, V, L, I, Q, N, D, E, S, T, or M
XXXLGWLCLLLLPIPLIVXVKXX

SEQ ID NO: 3 - protein, 23 aa, synthetic construct
  REGION 1..23: note = CD95 TMD R1E mutein
ESNLGWLCLLLLPIPLIVWVKRK

SEQ ID NO: 4 - protein, 23 aa, synthetic construct
  REGION 1..23: note = CD95 TMD R1Q mutein
QSNLGWLCLLLLPIPLIVWVKRK

SEQ ID NO: 5 - protein, 23 aa, synthetic construct
  REGION 1..23: note = CD95 TMD R1A mutein
ASNLGWLCLLLLPIPLIVWVKRK

SEQ ID NO: 6 - protein, 7 aa, Homo sapiens
KCKEEGS

SEQ ID NO: 7 - protein, 37 aa, Homo sapiens
CRCKPNFFCNSTVCEHCDPCTKCEHGIIKECTLTSNT

SEQ ID NO: 8 - protein, 43 aa, Homo sapiens
VPCQEGKEYTDKAHFSSKCRRCRLCDEGHGLEVEINCTRTQNT

SEQ ID NO: 9 - protein, 36 aa, Homo sapiens
QNLEGLHHDGQFCHKPCPPGERKARDCTVNGDEPDC

SEQ ID NO: 10 - protein, 46 aa, Homo sapiens
MLGIWTLLPLVLTSVARLSSKSVNAQVTDINSKGLELRKTVTTVET

SEQ ID NO: 11 - protein, 36 aa, Homo sapiens
EVQKTCRKHRKENQGSHESPTLNPETVAINLSDVDL

SEQ ID NO: 12 - protein, 88 aa, Homo sapiens
SKYITTIAGVMTLSQVKGFVRKNGVNEAKIDEIKNDNVQDTAEQVQLLRNWHQLHGKKEAYDTLIKDLKKANLCTLAEKIQTIILKDI

SEQ ID NO: 13 - protein, 17 aa, Homo sapiens
TSDSENSNFRNEIQSLV

SEQ ID NO: 14 - protein, 210 aa, synthetic construct
  REGION 1..210: note = CD95 truncated mutein
MLGIWTLLPLVLTSVARLSSKSVNAQVTDINSKGLELRKTVTTVETQSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQVQLLRNWHQLHGKKEAYDTLIKDLKKANLCTLAEKIQTIILKDITSDSENSNFRNEIQSLV

SEQ ID NO: 15 - protein, 334 aa, synthetic construct
  REGION 1..334: note = CD95 mutein R171Q
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSQSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQVQLLRNWHQLHGKKEAYDTLIKDLKKANLCTLAEKIQTIILKDITSDSENSNFRNEIQSLV

SEQ ID NO: 16 - DNA, 1008 bp, synthetic construct
  misc_feature 1..1008: note = CD95 TMD R171Q encoding
  source 1..1008: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatcccaatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 17 - DNA, 6551 bp, synthetic construct
  misc_feature 1..6551: note = AAV_CMV_CD95 TMD R171Q whole vector
  source 1..6551: mol_type = other DNA
gcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaagtgattatcggatcaactttgtatagaaaagttgggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatcccaatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctaggcggcc

SEQ ID NO: 18 - DNA, 1008 bp, synthetic construct
  misc_feature 1..1008: note = CD95 TMD R171E encoding
  source 1..1008: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccgagtctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 19 - DNA, 6511 bp, synthetic construct
  misc_feature 1..6511: note = AAV_CMV_CD95 TMD R171E whole vector
  source 1..6511: mol_type = other DNA
gcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccgagtctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctaggcggcc

SEQ ID NO: 20 - DNA, 8506 bp, synthetic construct
  misc_feature 1..8506: note = Lenti-CMV-CD95-TMD-R171E vector
  source 1..8506: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccgagtctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 21 - DNA, 1008 bp, synthetic construct
  misc_feature 1..1008: note = CD95 TMD R192E encoding
  source 1..1008: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaaggagaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 22 - DNA, 6547 bp, synthetic construct
  misc_feature 1..6547: note = AAV_CMV_CD95 TMD R192E whole vector
  source 1..6547: mol_type = other DNA
gcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagatatcggatcaactttgtatagaaaagttgggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaaggagaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctaggcgcggcc

SEQ ID NO: 23 - DNA, 8506 bp, synthetic construct
  misc_feature 1..8506: note = Lenti-CMV-CD95-TMD-R192E vector
  source 1..8506: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaaggagaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 24 - DNA, 1009 bp, synthetic construct
  misc_feature 1..1009: note = CD95 TMD K193E encoding
  source 1..1009: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagagagggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 25 - DNA, 6554 bp, synthetic construct
  misc_feature 1..6554: note = AAV_CMV_CD95 TMD K193E whole vector
  source 1..6554: mol_type = other DNA
gcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaggatcaactttgtatagaaaagttgggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagagagggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctaggcggccgcggccgcggcc

SEQ ID NO: 26 - DNA, 8506 bp, synthetic construct
  misc_feature 1..8506: note = Lenti-CMV-CD95-TMD-K193E vector
  source 1..8506: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagagaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 27 - DNA, 1008 bp, synthetic construct
  misc_feature 1..1008: note = CD95 TMD R192E/K193E encoding
  source 1..1008: mol_type = other DNA
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaaggaggaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 28 - DNA, 6551 bp, synthetic construct
  misc_feature 1..6551: note = AAV_CMV_CD95 TMD R192E/K193E whole vector
  source 1..6551: mol_type = other DNA
gcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaggatcaactttgtatagaaaagttgggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaaggaggaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctaggcggccgcgggcggcc

SEQ ID NO: 29 - DNA, 8506 bp, synthetic construct
  misc_feature 1..8506: note = Lenti-CMV-CD95-TMD-R192E/K193E vector
  source 1..8506: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctctggtggcggaggctcgggcggaggtgggtcgggtggcggcggatcaatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaaggaggaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 30 - protein, 334 aa, synthetic construct
  REGION 1..334: note = R171E single mutein (sM)
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSESNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQVQLLRNWHQLHGKKEAYDTLIKDLKKANLCTLAEKIQTIILKDITSDSENSNFRNEIQSLV

SEQ ID NO: 31 - protein, 334 aa, synthetic construct
  REGION 1..334: note = R192E/K193E double mutein (dM)
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSQSNLGWLCLLLLPIPLIVWVKEEEVQKTCRKHRKENQGSHESPTLNPETVAINLSDVDLSKYITTIAGVMTLSQVKGFVRKNGVNEAKIDEIKNDNVQDTAEQVQLLRNWHQLHGKKEAYDTLIKDLKKANLCTLAEKIQTIILKDITSDSENSNFRNEIQSLV

SEQ ID NO: 32 - protein, 334 aa, synthetic construct
  REGION 1..334: note = R171E/R192E/K193E triple mutein (tM)
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSESNLGWLCLLLLPIPLIVWVKEEEVQKTCRKHRKENQGSHESPTLNPETVAINLSDVDLSKYITTIAGVMTLSQVKGFVRKNGVNEAKIDEIKNDNVQDTAEQVQLLRNWHQLHGKKEAYDTLIKDLKKANLCTLAEKIQTIILKDITSDSENSNFRNEIQSLV

SEQ ID NO: 33 - protein, 23 aa, synthetic construct
  REGION 1..23: note = TM R22E/K23E double mutein
RSNLGWLCLLLLPIPLIVWVKEE

SEQ ID NO: 34 - protein, 23 aa, synthetic construct
  REGION 1..23: note = TM R1E/R22E/k23E triple mutein
ESNLGWLCLLLLPIPLIVWVKEE